(19) United States
(12) Patent Application Publication
(10) Pub. No.: US 2009/0233307 A1
(43) Pub. Date: Sep. 17, 2009

(54) METHODS FOR DETECTION OF SINGLE STRAND BREAKS IN DNA

(75) Inventors: Jun Nakamura, Chapel Hill, NC (US); April Luke, Chapel Hill, NC (US); James Swenberg, Pittsboro, NC (US)

Correspondence Address: MYERS BIGEL SIBLEY & SAJOVEC, PO BOX 37428, RALEIGH, NC, 27627, US

(73) Assignee: The University of North Carolina at Chapel Hill

(21) Appl. No.: 12/404,532

(22) Filed: Mar. 16, 2009

Related U.S. Application Data
(60) Provisional application No. 61/037,150, filed on Mar. 17, 2008.
(60) Provisional application No. 61/138,139, filed on Dec. 17, 2008.

(51) Int. Cl. C12Q 1/68; G01N 33/00

(52) U.S. Cl. 435/6; 436/94

(57) ABSTRACT

Methods are provided for detecting single strand breaks in DNA sample comprising: contacting the DNA with a neutral hydroxylamine, denaturing the DNA and detecting single strand breaks in the denatured DNA. Methods are also provided for the detecting single strand breaks in DNA from a cell, the methods comprising lysing the cell, denaturing the DNA from the lysed cell, wherein the DNA is contacted with a neutral hydroxylamine prior to and/or during the denaturing step and detecting single strand breaks in the DNA from the cell. In addition, methods are provided for distinguishing between true single strand breaks and artifactual single strand breaks resulting from cleaved alkaline labile sites.

### STATEMENT OF PRIORITY

This application claims the benefit, under 35 U.S.C. §119 (e), of U.S. Provisional Application No. 61/037,150, filed on Mar. 17, 2008, and U.S. Provisional Application No. 61/138,139 filed on Dec. 17, 2008, the entire contents of each of which are incorporated by reference herein.

### FEDERAL SUPPORT OF THE INVENTION

This invention was made with government support under grant number P42 ES005948 from the Department of Health and Human Services, National Institutes of Health, Superfund Basic Research Project NIEHS, grant number 5 P30 ES010126 from the Center for Environmental Health and Susceptibility and grant numbers ES07126 and T32-CA72319 from the National Institutes of Health. The United States government has certain rights in this invention.

### FIELD OF THE INVENTION

The present invention relates to methods for detecting real single strand breaks in DNA by reducing the false positive SSBs derived from alkali-labile sites cleaved during DNA denaturation.

### BACKGROUND OF THE INVENTION

Thousands of single strand breaks (SSBs) are believed to arise in cellular DNA daily from endogenous and exogenous sources as well as during DNA repair processes, such as base excision repair (BER) (Caldecott, DAN Repair 6, 443-453 (2007)). If unrepaired, these lesions threaten genetic integrity through their potential conversion to lethal double strand breaks during DNA replication. Therefore, repair of SSBs is critical to the cell. BER is a primary response pathway for the repair of deleterious DNA lesions including non-bulky DNA adducts, apurinic/apyrimidinic (AP) sites, and SSBs. In its simplest form, BER proceeds as follows: 1) removal of a damaged base through N-glycosylic bond cleavage by a mono-functional DNA glycosylase with AP site formation; 2) incision 5′ to the AP site by a Type II AP endonuclease, resulting in a SSB with 3′-hydroxyl and 5′-deoxyribosephosphate (5′-dRp) margins; 3) removal of the 5′-dRp and synthesis of the missing nucleotide by DNA polymerase beta (Pol β); 4) and strand ligation by a ligase (Fortini et al., DNA Repair 6:398-409 (2007)). Efficient BER is imperative because of the inadvertent generation of SSBs as pathway intermediates. While SSBs are one of the most frequent DNA lesions, well-characterized assays with a high specificity for SSBs are lacking.

The ability to detect SSBs has generated much interest for assessing genotoxicity. However, many of these approaches can generate confounded SSB data. A specific example is the single cell gel electrophoresis (comet) assay, which has gained much popularity throughout the past decade due to its ease and relative inexpensiveness (Anderson et al., Mutagenesis, 13, 67-73 (1998); Burlinson et al., Mutat. Res., 627, 31-35 (2007)). Similar to other SSB assays, the comet assay usually employs alkaline conditions (pH>13) to denature DNA. The presence of alkali-labile sites (ALSs), such as AP sites and oxidized AP sites, at high pH can lead to DNA strand cleavage. The resulting overestimation of SSB formation compromises the reliability of data obtained by the comet assay and other alkaline-based SSB assays (Anderson et al., Mutagenesis, 13, 67-73 (1998)). The potential for such variability has been observed in intra- and inter-laboratory studies (European Standards Committee on Oxidative DNA Damage, Free Radic. Biol. Med., 34, 1089-1099 (2003); Gedik et al., FASEB J., 19, 82-84 (2005)). The comet assay performed at pH 12.1-12.5 has been reported to detect SSBs without the contribution of artifactual SSBs from cleaved ALSs; however, this modification has also been reported to reduce the sensitivity of that assay (Speit et al., Toxicol. Lett., 146, 151-158 (2004)).

### SUMMARY OF THE INVENTION

The present invention is directed to methods of detecting single-strand breaks in DNA with reduced artifactual SSBs due to alkaline lability of the apurinic/apyrimidinic (AP) sites.

Accordingly, a first aspect of the invention is a method of detecting single strand breaks in a DNA sample comprising: contacting the DNA with a neutral hydroxylamine; denaturing the DNA; and detecting single strand breaks in the denatured DNA.

A second aspect of the invention is a method of detecting single strand breaks in DNA from a cell comprising: lysing the cell; denaturing the DNA from the lysed cell, wherein the DNA is contacted with a neutral hydroxylamine prior to and/or during the denaturing step; and detecting single strand breaks in the DNA from the cell.

A third aspect of the invention is a method of distinguishing between true single strand breaks and apurinic/apyrimidinic (AP) sites including oxidized AP sites in a DNA sample comprising: contacting a first portion of the DNA sample with a neutral hydroxylamine; denaturing the DNA of the first portion; detecting single strand breaks in the denatured DNA of the first portion; denaturing the DNA of a second portion of the DNA sample, wherein the second portion is not contacted with a neutral hydroxylamine; detecting single strand breaks in the denatured DNA of the second portion; and comparing the number of single strand breaks detected in the DNA of the first portion of the DNA sample with the number of single strand breaks detected in the second portion of the DNA sample, wherein the number of true single strand breaks in the DNA sample is represented by the number of single strand breaks detected in the first portion of the DNA sample; and the number of AP sites and oxidized AP sites present in the DNA sample is represented by the number of single strand breaks detected in the second portion of the DNA sample that is greater than the number of single strand breaks detected in the first portion of the DNA sample, thus distinguishing between true single strand breaks and AP sites and oxidized AP sites in the DNA sample.

A fourth aspect of the invention is a method of distinguishing between true single strand breaks and apurinic/apyrimidinic (AP) sites including oxidized AP sites in DNA from a cell sample comprising: detecting single strand breaks in the DNA of a first cell of the cell sample, wherein the first cell is lysed and the DNA from the lysed cell is denatured, further wherein the DNA is contacted with a neutral hydroxylamine prior to and/or during the denaturing step; detecting single strand breaks in the DNA of a second cell of the cell sample, wherein the second cell of the cell sample is lysed and the DNA from the lysed cell is denatured; and comparing the number of single strand breaks detected in the DNA of the first cell of the cell sample with the number of single strand breaks detected in the DNA of the second cell of the cell sample in which the cell was not contacted with a neutral hydroxylamine, wherein the number of true single strand breaks in the DNA of the cell(s) of the cell sample is represented by the number of single strand breaks detected in DNA of the first cell of the cell sample; and the number of AP sites and oxidized AP sites in the DNA of the cell(s) of the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized AP sites in the cell sample.

An additional aspect of the invention is a method of detecting single strand breaks in a DNA sample comprising: contacting the DNA with O-(tetrahydro-2H-pyran-2-yl)hydroxylamine (OTX); denaturing the DNA at a pH≧12.0: and detecting single strand breaks in the denatured DNA.

A further aspect of the invention is a method of detecting single strand breaks in DNA from a cell comprising: lysing the cell; denaturing the DNA from the lysed cell, wherein the DNA is contacted with O-(tetrahydro-2H-pyran-2-yl)hydroxylamine (OTX) prior to and/or the denaturing step and the denaturing of the DNA occurs at a pH≧12.0; and detecting single strand breaks in the DNA from the cell.

A still further aspect of the invention is method of distinguishing between true single strand breaks and apurinic/apyrimidinic (AP) sites including oxidized AP sites in a DNA sample comprising: contacting a first portion of the DNA sample with O-(tetrahydro-2H-pyran-2-yl)hydroxylamine (OTX); denaturing the DNA at a pH≧12.0; detecting single strand breaks in the denatured DNA of the first portion; denaturing the DNA of a second portion of the DNA sample at a pH≧12.0, wherein the second portion is not contacted with OTX; detecting single strand breaks in the denatured DNA of the second portion; and comparing the number of single strand breaks detected in the DNA of the first portion of the DNA sample with the number of single strand breaks detected in the second portion of the DNA sample, wherein the number of true single strand breaks in the DNA sample is represented by the number of single strand breaks detected in the first portion of the DNA sample; and the number of AP and oxidized AP sites present in the DNA sample is represented by the number of single strand breaks detected in the second portion of the DNA sample that is greater than the number of single strand breaks detected in the first portion of the DNA sample, thus distinguishing between true single strand breaks and AP and oxidized AP sites in the DNA sample.

Another aspect of the invention is a method of distinguishing between true single strand breaks and apurinic/apyrimidinic (AP) sites including oxidized AP sites in DNA from a cell sample comprising: detecting single strand breaks in the DNA of a first cell of the cell sample, wherein the first cell of the cell sample is lysed and the DNA from the lysed cell is denatured at a pH≧12.0, and further wherein the DNA is contacted with O-(tetrahydro-2H-pyran-2-yl)hydroxylamine (OTX) prior to and/or during the denaturing step; detecting single strand breaks in the DNA of a second cell of the cell sample, wherein the second cell of the cell sample is lysed and the DNA from the lysed cell(s) is denatured at a pH≧12.0; and comparing the number of single strand breaks detected in the DNA of the first cell of the cell sample with the number of single strand breaks detected in the DNA of the second cell of a the cell sample in which the cell was not contacted with OTX, wherein the number of true single strand breaks in the DNA of the cell(s) from the cell sample are represented by the number of single strand breaks detected in DNA of the first cell of the cell sample; and the number of AP and oxidized AP sites in the DNA of the cell(s) from the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized AP sites in the DNA from the cell sample.

### BRIEF DESCRIPTION OF THE FIGURES

FIG. 1 illustrates the extent of apurinic/apyrimidinic (AP) site cleavage by pH. Lane 1—No NaOH (Ø); Lanes 2-4—pH≦11.94; Lanes 5-8—pH 12.16-12.56; Lanes 9-12—pH≧12.67.

FIG. 2 shows OTX optimization for the protection of AP sites. Lane 1—Marker; Lane 2—No O-(tetrahydro-2H-pyran-2-yl)hydroxylamine (OTX) (Ø); Lanes 3-11—Heat/acid DNA+OTX (0.1, 0.3, 1, 3, 10, 30, 100, 300, and 1000 mM).

FIG. 3 shows single strand break (SSB) detection in DT40 and Pol β null cells exposed continuously to 1 mM methyl methanesulfonate (MMS) for up to 4 hours. (A) Real-time, indirect detection of SSB formation in the cell lines through the monitoring of intracellular NAD(P)H depletion. (B) Visualization of SSB formation in the cell lines through OTX-coupled alkaline gel electrophoresis (AGE) analysis. Even lanes—DT40 cells; Odd lanes with underline—Pol β null cells; Lane 1—Marker; Lanes 2-3—PBS control; Lanes 4-9—1 mM MMS exposure for 1-4 hours.

FIG. 4 shows AP site detection in DT40 and Pol β null cells exposed continuously to 1 mM MMS for up to 4 hours. (A) Numerical data of AP sites detected through the ASB assay. The mean values represent four independent measurements. Bars indicate SD. (B-C) AGE analysis with and without OTX protection. Even lanes—OTX protection; Odd lanes with underline—No OTX protection; Lane 1—Marker; Lanes 2-3—PBS control; Lanes 4-9—1 mM MMS exposure for 1-4 hours.

FIG. 5 shows data distinguishing SSBs arising from alkali-labile sites (ALSs) other than AP sites. Even lanes—PBS plus OTX treatment; Odd lanes with underline—MMS plus OTX treatment; Lane 1—Marker; Lanes 2-3—NaOH denaturation; Lanes 4-5—Piperidine denaturation.

FIG. 6 shows the difference in tail moments resulting from MMS exposure. Data is shown as mean±standard error of means.

FIG. 7 shows the difference in tail moments resulting from MMS exposure with (light grey) and without (dark grey) OTX Protection. Data is shown as mean±standard error of means. Statistical analysis: a=P<0.01, Dunnett test, comparison to the 0 hour incubation/no OTX protection control; b=P<0.0034, unpaired t test, comparison to the 0 hour incubation/no OTX protection control; c=P<0.001, unpaired t test, comparison to the 1 hour incubation/no OTX protection sample.

FIG. 8 shows the visual difference in comets resulting from MMS exposure without OTX protection (A) and with OTX protection (B). Comets resulted from a 10 minute exposure to 1 mM MMS.

### DETAILED DESCRIPTION

O-hydroxyalamines have been documented to react with the aldehyde group of AP and oxidized AP sites to create very stable complexes that are refractory to β-elimination by enzymatic activity (e.g., AP or dRp lyase) or by high pH (Horton et al., J. Biol. Chem. 275:2211-2218 (2000); Nakamura et al., Cancer Res. 58:222-225 (1998); Nakamura et al. Cancer Res., 59:2522-2526 (1999)). Methoxyamine has been used ex vivo as a BER inhibitor (Vermeulen et al., DNA Repair 6:202-212 (2007); Ukai et al., Genes Cells 11:111-121 (2006); Buschfort et al., Cancer Res., 57:651-658 (1997)) and in vitro as a stabilizer of AP sites for detect of damaged bases (e.g. N7-methylpurines) (Bartlett et al., Mutat Res. 255:247-256 (1991); Scicchitano et al., Proc. Natl. Acad. Sci. USA. 86:3050-3054 (1989)) or AP sites (Fortini et al., Mutat Res. 236:129-137 (1990); Talpaert-Borlé, Mutat Res. 181:45-56 (1987)). However, the acidic methoxyamine must be precisely neutralized before and during use to prevent the artifactual formation of AP sites and SSBs under acidic and basic conditions. respectfully. Furthermore, the neutralization of methoxyamine can lead to the production of high salt contamination. The presence of salt retards DNA migration during electrophoresis ultimately leading to an underestimate of SSB formation (Fairbairn et. al., Mutation Res. 339:37-59 (1995); Olive et. al., Exp. Cell Res. 198:259-267 (1992)). As an alternative to methoxyamine, neutral O-hydroxylamines, such as O-(tetrahydro-2H-pyran-2-yl)hydroxylamine (OTX), do not require neutralization and thus, circumvent the salt contamination observed with acidic methoxyamine. Accordingly, the present invention provides a novel neutral hydroxylamine-coupled alkaline gel electrophoresis method for the specific detection of true SSBs.

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

### Definitions.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. Thus, the term “consisting essentially of” when used in a claim of this invention is not intended to be interpreted to be equivalent to “comprising.”

As used herein a “neutral hydroxylamine” refers to a hydroxylamine compounds that do not require neutralization prior to their use with this invention. Examples of such compounds include, but are not limited to 2-(2-(2-aminooxy-ethoxy)-ethoxy)-ethyl-hydroxylamine picrate, O-trityhydroxylamine, O-(tert-butyldimethylsilyl)hydroxylamine, O-(trimethylsilyl)hydroxylamine, O-(tetrahydro-2H-pyran-2-yl)hydroxylamine, aldehyde reactive probe and the like. The pH range of a neutral hydroxylamines is in a range of between about 6 to about 9. Thus, neutral hydroxylamines do not include methoxyamine hydrochloride, and the like, which require neutralization prior to use.

“True single strand breaks” (SSB) as used herein refer to single strand breaks in DNA that are induced by endogenous and exogenous agents as opposed to single strand breaks resulting from the creation and scission of alkali-labile sites (ALSs) during DNA preparation and denaturation.

“Alkali-labile sites” as used herein refers to and includes, but is not limited to, abasic sites also referred to as apurinic/apyrimidinic sites (AP) AP sites include, but are not limited to “regular” AP sites and oxidized AP sites. Regular AP sites are formed through enzymatic (i.e., DNA glycosylases) or acid catalyzed depurination or depyrimidination in which the DNA bases are hydrolytically removed. Oxidized AP sites are formed via two pathways both initiated by H atom abstraction from the 1′ or 4′ carbon of the DNA deoxyribose. (See, Demple et al. Oncogene 21(58):8926-8934 (2002); Dedon, P., Chem. Res. Toxicol. 21:206-219 (2008))

All publications, patent applications, patents and other references cited herein are incorporated by reference in their entireties for the teachings relevant to the sentence and/or paragraph in which the reference is presented.

A first aspect of the present invention is a method of detecting single strand breaks in a DNA sample comprising: contacting the DNA with a neutral hydroxylamine; denaturing the DNA; and detecting single strand breaks in the denatured DNA. In some embodiments, the DNA is contacted with a neutral hydroxylamine prior to and/or during the denaturing step.

A neutral hydroxylamine of the present invention includes, but is not limited to, 2-(2-(2-aminooxy-ethoxy)-ethoxy)-ethyl-hydroxylamine picrate, O-trityhydroxylamine, O-(tert-butyldimethylsilyl)hydroxylamine, O-(trimethylsilyl)hydroxylamine, O-(tetrahydro-2H-pyran-2-yl)hydroxylamine and aldehyde reactive probe. In some embodiments of the invention, the neutral hydroxylamine is O-(tetrahydro-2H-pyran-2-yl)hydroxylamine.

An additional aspect of the invention is a method of detecting single strand breaks in a DNA sample comprising: contacting the DNA with O-(tetrahydro-2H-pyran-2-yl)hydroxylamine (OTX); denaturing the DNA at a pH≧12.0; and detecting single strand breaks in the denatured DNA.

In some embodiments of the invention, the neutral hydroxylamine is provided at a concentration in a range from about 0.1 mM to about 1000 mM. In other embodiments, the neutral hydroxylamine is provided at a concentration in a range from about 3 mM to about 300 mM. In still further embodiments, the neutral hydroxylamine is provided at a concentration in a range from about 3 mM to about 100 mM. Thus, the neutral hydroxylamine can be provided at a concentration of about 0.1 mM, 0.2 mM, 0.3 mM, 0.4 mM, 0.5 mM, 0.6 mM, 0.7 mM, 0.8 mM, 1 mM, 2, mM, 3 mM, 4 mM, 5 mM, 6 mM, 7 mM, 8 mM, 9 mM, 10 mM, 11 mM, 12, mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, 500 mM, 510 mM, 520 mM, 530 mM, 540 mM, 550 mM, 560 mM, 570 mM, 580 mM, 590 mM, 600 mM, 610 mM, 620 mM, 630 mM, 640 mM, 650 mM, 660 mM, 670 mM, 680 mM, 690 mM, 700 mM, 710 mM, 720 mM, 730 mM, 740 mM, 750 mM, 760 mM, 770 mM, 780 mM, 790 mM, 800 mM, 810 mM, 820 mM, 830 mM, 840 mM, 850 mM, 860 mM, 870 mM, 880 mM, 890 mM, 900 mM, 910 mM, 920 mM, 930 mM, 940 mM, 950 mM, 960 mM, 970 mM, 980 mM, 990 mM, or 1000 mM, and the like.

In other embodiments, the neutral hydroxylamine can be provided at a concentration in a range from 1 mM to 3 mM, 1 mM to 10 mM, 1 mM to 50 mM, 3 mM to 10 mM, 3 mM to 50 mM, 5 mM to 50 mM, 10 mM to 30 mM, 10 mM to 50 mM, 20 mM to 50 mM, 30 mM to 50 mM, 40 mM to 50 mM, 1 mM to 100 M, 5 mM to 100 mM, 10 mM to 100 mM, 20 mM to 100 mM, 30 mM to 100 mM, 40 mM to 100 mM, 50 mM to 100 mM, 60 mM to 100 mM, 70 mM to 100 mM, 80 mM to 100 mM, 90 mM to 100 mM, 1 mM to 200 mM, 3 mM to 200 mM, 5 mM to 200 mM, 10 mM to 200 mM, 20 mM to 200 mM, 30 mM to 200 mM, 40 mM to 200 mM, 50 mM to 200 mM, 60 mM to 200 mM, 70 mM to 200 mM, 80 mM to 200 mM, 90 mM to 200 mM, 100 mM to 200 mM, 150 mM to 200 mM, 1 mM to 300 mM, 5 mM to 300 mM, 10 mM to 300 mM, 20 mM to 300 mM, 30 mM to 300 mM, 40 mM to 300 mM, 50 mM to 300 mM, 60 mM to 300 mM, 70 mM to 300 mM, 80 mM to 300 mM, 90 mM to 300 mM, 100 mM to 300 mM, 150 mM to 300 mM, 200 mM to 300 mM, 250 mM to 300 mM, 1 mM to 400 mM, 3 mM to 400 mM, 5 mM to 400 mM, 10 mM to 400 mM, 20 mM to 400 mM, 30 mM to 400 mM, 40 mM to 400 mM, 50 mM to 400 mM, 60 mM to 400 mM, 70 mM to 400 mM, 80 mM to 400 mM, 90 mM to 400 mM, 100 mM to 400 mM, 150 mM to 400 mM, 200 mM to 400 mM, 250 mM to 400 mM, 300 mM to 400 mM, 350 mM to 400 mM, 1 mM to 500 mM, 3 mM to 500 mM, 5 mM to 500 mM, 10 mM to 500 mM, 20 mM to 500 mM, 30 mM to 500 mM, 40 mM to 500 mM, 50 mM to 500 mM, 60 mM to 500 mM, 70 mM to 500 mM, 80 mM to 500 mM, 90 mM to 500 mM, 100 mM to 500 mM, 150 mM to 500 mM, 200 mM to 500 mM, 250 mM to 500 mM, 300 mM to 500 mM, 350 mM to 500 mM, 400 mM to 500 mM, 450 mM to 500 mM, 10 mM to 600 mM, 50 mM to 600 mM, 100 mM to 600 mM, 150 mM to 600 mM, 200 mM to 600 mM, 250 mM to 600 mM, 300 mM to 600 mM, 350 mM to 600 mM, 400 mM to 600 mM, 450 to 600 mM, 500 mM to 600 mM, 550 mM to 600 mM, 10 mM to 700 mM, 50 mM to 700 mM, 100 mM to 700 mM, 150 mM to 700 mM, 200 mM to 700 mM, 250 mM to 700 mM, 300 mM to 700 mM, 350 mM to 700 mM, 400 mM to 700 mM, 450 to 700 mM, 500 mM to 700 mM, 550 mM to 700 mM, 600 mM to 700 mM, 650 mM to 700 mM, 10 mM to 800 mM, 50 mM to 800 mM, 100 mM to 800 mM, 150 mM to 800 mM, 200 mM to 800 mM, 250 mM to 800 mM, 300 mM to 800 mM, 350 mM to 800 mM, 400 mM to 800 mM, 450 to 800 mM, 500 mM to 800 mM, 550 mM to 800 mM, 600 mM to 800 mM, 650 mM to 800 mM, 700 mM to 800 mM, 750 mM to 800 mM, 10 mM to 900 mM, 50 mM to 900 mM, 100 mM to 900 mM, 150 mM to 900 mM, 200 mM to 900 mM, 250 mM to 900 mM, 300 mM to 900 mM, 350 mM to 900 mM, 400 mM to 900 mM, 450 to 900 mM, 500 mM to 900 mM, 550 mM to 900 mM, 600 mM to 900 mM, 650 mM to 900 mM, 700 mM to 900 mM, 750 mM to 900 mM, 800 mM to 900 mM, 850 mM to 900 mM, 10 mM to 1000 mM, 50 mM to 1000 mM, 100 mM to 1000 mM, 150 mM to 1000 mM, 200 mM to 1000 mM, 250 mM to 1000 mM, 300 mM to 1000 mM, 350 mM to 1000 mM, 400 mM to 1000 mM, 450 to 1000 mM, 500 mM to 1000 mM, 550 mM to 1000 mM, 600 mM to 1000 mM, 650 mM to 1000 mM, 700 mM to 1000 mM, 750 mM to 1000 mM, 800 mM to 1000 mM, 850 mM to 1000 mM, 900 mM to 1000 mM, or 950 mM to 1000 mM, and the like.

In some embodiments of the invention, the contacting comprises contacting the DNA with the neutral hydroxylamine at a temperature in a range from about 0° C. to about 60° C., a pH in a range from about 6.0 to about 9.0, and for a period of time in a range from about 1 min to about two days.

Thus, in some embodiments, the contacting comprises contacting the DNA with a neutral hydroxylamine at a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C. or 60° C., and the like. In other embodiments, the contacting comprises contacting the DNA with a neutral hydroxylamine at a temperature in a range from about 0° C. to 20° C., 0° C. to 30° C., 0° C to 40° C., 0° C. to 50° C., 0° C. to 60° C., 5° C. to 10° C., 5° C. to 20° C., 5° C. to 30° C., 5° C. to 40° C., 5° C. to 50° C., 5° C. to 60° C., 10° C. to 20° C., 10° C. to 30° C., 10° C. to 40° C., 10° C. to 50° C., 10° C. to 60° C., 15° C. to 20° C., 15° C. to 30° C., 15° C. to 40° C., 15° C. to 50° C., 15° C. to 60° C., 20° C. to 30° C., 20° C. to 40° C., 20° C. to 50° C., 20° C. to 60° C., 25° C. to 30° C., 25° C. to 40° C., 25° C. to 50° C., 25° C. to 60° C., 30° C. to 35° C., 30° C. to 40° C., 30° C. to 50° C., 30° C. to 60° C., 35° C. to 40° C., 35° C. to 50° C., 35° C. to 60° C., 40° C. to 45° C., 40° C. to 50° C., 40° C. to 60° C., 45° C. to 50° C., 45° C. to 60° C., 50° C. to 55° C., 50° C. to 60° C., or 55° C. to 60° C., and the like

In further embodiments, the contacting comprises contacting the DNA with a neutral hydroxylamine at a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0 and the like. In other embodiments, the contacting comprises contacting the DNA with a neutral hydroxylamine at a pH in a range from about 6.0 to 6.5, 6.0 to 7.0, 6.0 to 7.5, 6.0 to 8.0, 6.0 to 8.5, 6.0 to 9.0, 6.5 to 7.0, 6.5 to 7.5, 6.5 to 8.0, 6.5 to 8.5, 6.5 to 9.0, 7.0 to 7.5, 7.0 to 8.0, 7.0 to 8.5, 7.0 to 9.0, 7.5 to 8.0, 7.5 to 8.5, 7.5 to 9.0, 8.0 to 8.5, 8.0 to 9.0, or 8.5 to 9.0, and the like. In some further embodiments, the contacting comprises contacting the DNA with a neutral hydroxylamine at a pH in a range from about 7.2 to 7.3, 7.2 to 7.4, 7.2 to 7.5, 7.3 to 7.4, 7.3 to 7.5, 7.3 to 7.6, 7.4 to 7.5, or 7.4 to 7.6, and the like.

In still further embodiments, the contacting comprises contacting the DNA with a neutral hydroxylamine for a period of time of about 1 min, 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, or 120 min, 3 hr, 4 hr, 5 hr, 6 hr, 7 hr, 8 hr, 9 hr, 10 hr, 11 hr, 12 hr, one day, two days, and the like. In other embodiments, the contacting comprises contacting the DNA with a neutral hydroxylamine for a period of time in a range from about 1 min to 10 min, 5 min to 10 min, 10 min to 15 min, 10 min to 20 min, 10 min to 25 min, 10 min to 30 min, 15 min to 20 min, 15 min to 25 min, 15 min to 30 min, 20 min to 30 min, 30 min to 35 min, 30 min to 40 min, 30 min to 45 min, 30 min to 50 min 30 min to 55 min, 30 min to 60 min, 30 min to 65 min, 30 min to 70 min, 30 min to 75 min, 30 min to 80 min, 30 min to 85 min, 30 min to 90 min, 30 min to 95 min 30 min to 100 min, 30 min to 105 min, 30 min to 110 min, 30 min to 115 min, 45 min to 50 min, 45 min to 55 min, 45 min to 60 min, 45 min to 65 min, 45 min to 70 min, 45 min to 75 min, 45 min to 80 min, 45 min to 85 min, 45 min to 90 min, 45 min to 95 min, 45 min to 100 min, 45 min to 105 min, 45 min to 110 min 45 min to 115 min, 45 min to 120 min, 60 min to 65 min, 60 min to 70 min, 60 min to 75 min, 60 min to 80 min, 60 min to 85 min, 60 min to 90 min, 60 min to 95 min, 60 min to 100 min, 60 min to 105 min, 60 min to 110 min, 60 min to 115 min, 60 min to 120 min, 75 min to 80 min, 75 min to 85 min, 75 min to 90 min, 75 min to 95 min, 75 min to 100 min, 75 min to 105 min, 75 min to 110 min, 75 min to 115 min, 75 min to 120 min, 90 min to 95 min, 90 min to 100 min, 90 min to 105 min, 90 min to 110 min, 90 min to 115 min, 90 min to 120 min, or 105 min to 110 min, 105 min to 115 min, 105 min to 120 min, 2 hr to 4 hr, 2 hr to 8 hr, 2 hr to 12 hr, 2 hr to 1 day, 2 hr to 2 days, and the like.

Methods for denaturing DNA are known in the art. For example, DNA can be denatured by treatment with an alkaline buffer. An example of an alkaline buffer includes, but is not limited to, 100 mM NaOH, 30 mM OTX, 50 mM HEPES at pH 12.8. The denaturing of the DNA can take place in the presence of the neutral hydroxylamine or alternatively, the excess neutral hydroxylamine can be removed prior to the denaturing of the DNA.

In some embodiments of the invention, the denaturing of the DNA occurs at a pH≧12.0. In other embodiments, the denaturing of the DNA occurs at a pH≧13.0. Thus, the denaturing of the DNA occurs at a pH of 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0, and the like.

Thus, an additional aspect of the invention is a method of detecting single strand breaks in a DNA sample comprising: contacting the DNA with O-(tetrahydro-2H-pyran-2-yl)hydroxylamine (OTX); denaturing the DNA at a pH≧12.0; and detecting single strand breaks in the denatured DNA. In some embodiments, the DNA is contacted with OTX prior to and/or during the denaturing step.

Single strand breaks in the denatured DNA can be detected using methods known in the art including, but not limited to, alkaline gel electrophoresis (See., e.g., Drouin et al., Technologies for Detection of DNA Damage and Mutations (G. P. Pfeifer, ed.), Plenum Press, New York, N.Y., pp. 37-43 (1996); Gabelova et al. Neoplasma 44(6):380-388 (1997)). In some embodiments of the invention, alkaline gel electrophoresis comprises preparation of an agarose gel. The DNA samples are contacted with a neutral hydroxylamine prior to or during the denaturation step. Following denaturation the samples are loaded onto the gel and subjected to electrophoresis. The gels are then stained with for visualization of the DNA. In some embodiments, the DNA sample that has been contacted with a neutral hydroxylamine is denatured after it is loaded onto the gel (i.e., denaturing takes place in the sample well).

Image analysis software can be used to visualize the DNA in the gels. By comparison to standards, the amount of DNA and number of single strand breaks can be quantified. In some embodiments, the quantification utilizes DNA markers to mathematically estimate single strand breaks (See, e.g., Sutherland et al. Biochemistry 42:3375-3384 (2003)).

A further aspect of the invention is a method of detecting single strand breaks in DNA from a cell comprising: lysing the cell; denaturing the DNA from the lysed cell, wherein the DNA is contacted with a neutral hydroxylamine prior to and/or during the denaturing step; and detecting single strand breaks in the DNA from the cell.

The neutral hydroxylamines useful with the present invention and concentrations thereof are set forth above.

Thus, an additional aspect of the invention is a method of detecting single strand breaks in DNA from a cell comprising: lysing the cell; denaturing the DNA from the lysed cell, wherein the DNA is contacted with O-(tetrahydro-2H-pyran-2-yl)hydroxylamine (OTX) prior to and/or the denaturing step and the denaturing of the DNA occurs at a about pH≧12.0; and detecting single strand breaks in the DNA from the cell.

The cells to be used with the methods of this invention can be derived from in vitro cell culture or they can be derived directly from a subject (in vivo). Subjects from which cells are derived can any cellular organism. Thus, subjects of the invention include any uni- or any multicellular organism, and both prokaryotic and eukaryotic organisms.

Methods for lysing a cell are known in the art. For example, a cell can be lysed using a concentrated salt solution or a detergent. (Singh et al., Experimental Cell Research 175:184-191 (1988); Moneef et al., British J. of Cancer 89:2271-2276 (2003)).

As described above, the DNA from the cell is contacted with the neutral hydroxylamine prior to and/or during the denaturing step. Thus, in some embodiments, the neutral hydroxylamine is provided during the DNA denaturing step only. In other embodiments, the neutral hydroxylamine is provided during the denaturing step and in one or more of steps prior to the DNA denaturing step including, but not limited to, the lysing of the cells. In other embodiments, the neutral hydroxylamine is provided in the DNA denaturing step and all steps prior to the denaturing step. Thus, the neutral hydroxylamine may be provided in one or more of the buffers to be used with the methods of the present invention.

In additional embodiments of the invention, in order to detect the single strand breaks in the DNA of a cell(s), the cell(s) is suspended in low melt agarose and the cell(s) is then lysed as described above. The DNA that is released from the lysed cell is denatured according to methods known in the art including, but not limited to, exposure to alkaline conditions, such as an alkaline buffer. The denatured DNA in the low melt agarose gel is then subjected to electrophoresis. Following electrophoresis, the gel neutralized and stained with a DNA-specific dye. Image analysis software can be used to visualize the DNA (e.g., Comet Software IV).

In further embodiments of the invention, the method of detecting single strand breaks in DNA from a cell comprises the single cell gel electrophoresis assay or the “comet assay,” wherein the DNA is contacted with a neutral hydroxylamine prior to and/or during the DNA denaturing step. The single cell gel electrophoresis assay or comet assay is well known in the art (Singh et al., Experimental Cell Research 175:184-191 (1988)); Moneef et al., British J. of Cancer 89:2271-2276 (2003); Olive et al., Cancer Research 51:4671-4676 (1991); Olive et al. Nat. Protoc. 1(1):23-29 (2006)).

In some embodiments of the present invention, a comet assay comprises suspending a cell into low-melt agarose cast onto a microscope slide or other similar surface. The cell is then lysed to release the DNA, typically by immersing the gel into a concentrated salt solution or a detergent. Following lysis, the slides are washed and the DNA in the gel is denatured and subjected to electrophoresis. The slides are washed and the pH reduced to about 7. The DNA is stained and visualized using image analysis software.

As discussed above, the neutral hydroxylamine can be provided in the DNA denaturing step only but, in addition, may also be provided during cell preparation including, but not limited to, the cell washing and cell lysis steps.

A further aspect of the invention is a method of distinguishing between true single strand breaks and apurinic/apyrimidinic (AP) sites including oxidized AP sites in a DNA sample comprising: contacting a first portion of the DNA sample with a neutral hydroxylamine; denaturing the DNA of the first portion; detecting single strand breaks in the denatured DNA of the first portion; denaturing the DNA of a second portion of the DNA sample, wherein the second portion is not contacted with a neutral hydroxylamine; detecting single strand breaks in the denatured DNA of the second portion; and comparing the number of single strand breaks detected in the DNA of the first portion of the DNA sample with the number of single strand breaks detected in the second portion of the DNA sample, wherein the number of true single strand breaks in the DNA sample is represented by the number of single strand breaks detected in the first portion of the DNA sample; and the number of AP and oxidized AP sites present in the DNA sample is represented by the number of single strand breaks detected in the second portion of the DNA sample that is greater than the number of single strand breaks detected in the first portion of the DNA sample, thus distinguishing between true single strand breaks and AP and oxidized AP sites in the DNA sample.

Thus, as disclosed herein, by comparing the number of single strand breaks detected in a first portion of a DNA sample treated with a neutral hydroxylamine to the number of single strand breaks detected in a second portion of a DNA sample that has not been treated with a neutral hydroxylamine, true single strand breaks can be distinguished from AP and oxidized AP sites in DNA from a cell in a cell sample. As set forth above, the number of true single strand breaks is represented by the number of single strand breaks detected in the DNA sample which has been treated with the neutral hydroxylamine and the number of AP and oxidized AP sites in the DNA sample is represented by the number of single strand breaks detected in the portion of the DNA sample which was not treated with the neutral hydroxylamine minus the number of true single strand breaks detected in the portion of the DNA sample treated with the hydroxylamine.

A further aspect of the invention is method of distinguishing between true single strand breaks and apurinic/apyrimidinic (AP) sites including oxidized AP sites in a DNA sample comprising: contacting a first portion of the DNA sample with O-(tetrahydro-2H-pyran-2-yl)hydroxylamine (OTX); denaturing the DNA at a about pH≧12.0; detecting single strand breaks in the denatured DNA of the first portion; denaturing the DNA of a second portion of the DNA sample at a pH≧12.0, wherein the second portion is not contacted with OTX; detecting single strand breaks in the denatured DNA of the second portion; and comparing the number of single strand breaks detected in the DNA of the first portion of the DNA sample with the number of single strand breaks detected in the second portion of the DNA sample, wherein the number of true single strand breaks in the DNA sample is represented by the number of single strand breaks detected in the first portion of the DNA sample; and the number of AP and oxidized AP sites present in the DNA sample is represented by the number of single strand breaks detected in the second portion of the DNA sample that is greater than the number of single strand breaks detected in the first portion of the DNA sample, thus distinguishing between true single strand breaks and AP and oxidized AP sites in the DNA sample.

According to the methods of the present invention, the contacting of the first portion of the DNA sample with a neutral hydroxylamine is carried out as described above. In addition, the neutral hydroxylamines of this invention and methods of denaturing DNA are as described above. Further, methods of detecting the single strand breaks are described above.

An additional aspect of the invention is a method of distinguishing between true single strand breaks and apurinic/apyrimidinic (AP) sites including oxidized AP sites in DNA from a cell sample comprising: detecting single strand breaks in the DNA of a first cell of the cell sample, wherein the first cell is lysed and the DNA from the lysed cell is denatured, further wherein the DNA is contacted with a neutral hydroxylamine prior to and/or during the denaturing step; detecting single strand breaks in the DNA of a second cell of the cell sample, wherein the second cell of the cell sample is lysed and the DNA from the lysed cell is denatured; and comparing the number of single strand breaks detected in the DNA of the first cell of the cell sample with the number of single strand breaks detected in the DNA of the second cell of the cell sample in which the cell was not contacted with a neutral hydroxylamine, wherein the number of true single strand breaks in the DNA of the cell(s) of the cell sample is represented by the number of single strand breaks detected in DNA of the first cell of the cell sample; and the number of AP and oxidized AP sites in the DNA of the cell(s) of the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized AP sites in DNA from the cell sample.

Thus, as disclosed herein, by comparing the number of single strand breaks detected in the DNA of a first cell of a cell sample treated with a neutral hydroxylamine to the number of single strand breaks detected in the DNA of a second cell of a cell sample that has not been treated with a neutral hydroxylamine true single strand breaks can be distinguished from AP and oxidized AP sites in DNA from a cell in a cell sample. As set forth above, the number of true single strand breaks is represented by the number of single strand breaks detected in the DNA of the cell treated with the neutral hydroxylamine and the number of AP and oxidized AP sites in the DNA of the cells in the cell sample is represented by the number of single strand breaks detected in the cells of the cell sample that were not contacted with the neutral hydroxylamine minus the number of true single strand breaks detected in the cells of the cell sample treated with the hydroxylamine.

Another aspect of the invention is a method of distinguishing between true single strand breaks and apurinic/apyrimidinic (AP) sites including oxidized AP sites in DNA from a cell sample comprising: detecting single strand breaks in the DNA of a first cell of the cell sample, wherein the first cell of the cell sample is lysed and the DNA from the lysed cell is denatured at a about pH≧12.0, and further wherein the DNA is contacted with O-(tetrahydro-2H-pyran-2-yl)hydroxylamine (OTX) prior to and/or during the denaturing step; detecting single strand breaks in the DNA of a second cell of the cell sample, wherein the second cell of the cell sample is lysed and the DNA from the lysed cell(s) is denatured at a pH≧12.0; and comparing the number of single strand breaks detected in the DNA of the first cell of the cell sample with the number of single strand breaks detected in the DNA of the second cell of a the cell sample in which the cell was not contacted with OTX, wherein the number of true single strand breaks in the DNA of the cell(s) from the cell sample are represented by the number of single strand breaks detected in DNA of the first cell of the cell sample; and the number of AP and oxidized AP sites in the DNA of the cell(s) from the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized sites in DNA from the cell sample.

The contacting of the cells of the cell sample with a neutral hydroxylamine is carried out as described above. In addition, the neutral hydroxylamines and cells of this invention and methods of denaturing DNA are as described above. Further, methods of detecting the single strand breaks are described above.

In further embodiments, the present invention provides the methods of detecting DNA repair intermediates; more specifically for detection of the DNA repair intermediates which are AP sites and single strand breaks introduced during DNA repair processes. Therefore, this method is useful to determine the ability to fix DNA repair intermediates.

In further embodiments of the invention, methods for detecting true double strand breaks are provided utilizing a neutral hydroxylamine-coupled gel electrophoresis assay. Clustered DNA damage with labile DNA lesions can introduce double strand breaks during DNA preparation. (Hada et al. J. Radiat. Res. 49:203-210 (2008)). Such DNA lesions include abasic sites, which can be caused by factors including, but not limited to, oxidative stress and/or radiation. Abasic sites can be stabilized by a neutral hydroxylamine to prevent formation of double strand breaks during DNA preparation. This method provides an estimate of true double strand breaks.

Accordingly, further aspects of the present invention provide methods of detecting double strand breaks in a DNA sample comprising: contacting the DNA with a neutral hydroxylamine; and detecting double strand breaks in the DNA. In some embodiments of the present invention, the DNA is contacted with a neutral hydroxylamine prior to and/or during the neutral gel electrophoresis step. In some embodiments, the neutral hydroxylamine is O-(tetrahydro-2H-pyran-2-yl)hydroxylamine (OTX).

The neutral hydroxylamine compounds of the invention and their concentrations as well as other conditions for the assays including temperature, pH, and contacting time are set forth above.

Other aspects of the invention provide methods of detecting double strand breaks in DNA from a cell comprising: lysing the cell; contacting the DNA from the lysed cell with a neutral hydroxylamine prior to and/or during the neutral gel electrophoresis step (e.g., pulsed field gel electrophoresis and neutral comet assay); and detecting double strand breaks in the DNA from the cell. In some embodiments, the neutral hydroxylamine is O-(tetrahydro-2H-pyran-2-yl)hydroxylamine. The neutral hydroxylamine compounds of the invention and their concentrations as well as other conditions for the assays including temperature, pH, and contacting time are set forth above.

Clustered or closely spaced bi-stranded abasic sites in DNA are cleaved by monamines and polyamines resulting in double strand breaks (Georgakilas et al. Nucleic Acids Res. 30:2800-2808 (2002); Steullet et al. Bioorganic & Medicinal Chemistry 7 (11):2531-2540 (1999)). Thus, assaying for double strand breaks in DNA after treatment with monoamines and polyamines can provide an estimate of the number of clustered or closely spaced bi-stranded abasic sites in DNA. However, not all double strand breaks result from clustered abasic sites. In order to determine a more true estimate of the clustered abasic sites, the abasic sites in the DNA can be cleaved by treatment with a monoamine and/or polyamine prior to reaction of abasic sites with a neutral hydroxylamine. A portion of the sample is also first treated with a neutral hydroxylamine followed by treatment with the monoamine and/or polyamine. The difference in double strand breaks between these two samples provides a more true estimate of the clustered or closely spaced bi-stranded abasic sites in DNA. Thus, in a sample, the number of double strand breaks detected in the portion of the sample that is treated with the monoamine/polyamine prior to treatment with the neutral hydroxylamine that is larger than the number of double strand breaks in the portion treated with the neutral hydroxylamine prior to treatment with the monoamine/polyamine represents the clustered or closely spaced bi-stranded abasic sites.

Accordingly, in some embodiments, methods are provided for detecting clustered or closely spaced bi-stranded abasic sites in a DNA sample, the methods comprising: contacting a first portion of the DNA sample with a monoamine and/or polyamine; then contacting the monoamine/polyamine treated DNA of the first portion with a neutral hydroxylamine: detecting double strand breaks in the DNA of the first portion; contacting the DNA of a second portion of the DNA sample with a neutral hydroxylamine, and then contacting the hydroxylamine treated DNA of the second portion with a monoamine and/or polyamine; detecting double strand breaks in the DNA of the second portion; and comparing the number of double strand breaks detected in the DNA of the first portion of the DNA sample with the number of double strand breaks detected in the second portion of the DNA sample, wherein the number of closely space bi-strand abasic sites in the DNA sample is represented by the number of double strand breaks detected in the first portion of the DNA sample that is greater than the number of double strand breaks detected in the second portion of the DNA; thus, detecting clustered or closely spaced bi-stranded abasic sites in a DNA sample and distinguishing between double strand breaks resulting from closely spaced bi-strand abasic sites cleaved by the monoamines and/or polyamine and double strand breaks resulting from any other cause in the DNA sample. In some embodiments, the neutral hydroxylamine is O-(tetrahydro-2H-pyran-2-yl)hydroxylamine (OTX).

Further embodiments of the present invention provide methods for detecting clustered or closely spaced bi-stranded abasic sites in DNA from a cell sample, the method comprising: lysing a first cell of the cell sample; contacting the DNA from the first cell with a monoamine and/or polyamine; contacting the monoamine/polyamine treated DNA of the first cell with a neutral hydroxylamine; detecting double strand breaks in the DNA of the first cell of the cell sample, lysing a second cell of the cell sample; contacting the DNA from the second cell with a neutral hydroxylamine; then contacting the neutral hydroxylamine treated DNA of the second cell with a monoamine and/or polyamine; detecting double strand breaks in the DNA of the second cell of the cell sample; and comparing the number of double strand breaks detected in the DNA of the first cell of the cell sample with the number of double strand breaks detected in the DNA of the second cell of the cell sample, wherein the number of closely spaced bi-stranded abasic sites in the DNA of the cell sample is represented by the number of double strand breaks detected in DNA of the first cell of the cell sample that is greater than the number of double strand breaks detected in the DNA of the second cell of the cell sample, thus, detecting closely spaced bi-stranded abasic sites in a cell sample and distinguishing between double strand breaks resulting from closely spaced bi-strand abasic sites cleaved by the monoamines and/or polyamine and double strand breaks resulting from any other cause in the cell sample. In some embodiments, the neutral hydroxylamine is O-(tetrahydro-2H-pyran-2-yl)hydroxylamine (OTX).

The DNA from the first cell can be contacted with a neutral hydroxylamine prior to and/or during the neutral gel electrophoresis step.

A monoamine or polyamine of the present invention, includes, but is not limited to, piperidine, spermine, spermidine, and putrescine, and any combination thereof.

In some embodiments of the present invention, the monoamine and/or polyamine is provided at a concentration in a range from about 1 μM to about 1000 mM. Thus, in some embodiments, the monoamine and/or polyamine is provided at a concentration of about 10 μM to about 1000 mM, 10 μM to about 500 mM, about 100 μM to about 1000 mM, about 100 μM to about 500 mM, about 1 mM to about 1000 mM, about 1 mM to about 500 mM, about 10 mM to about 1000 mM, about 10 mM to about 100 mM, about 10 mM to about 500 mM, about 100 mM to about 1000 mM, about 100 mM to about 500 mM, about 500 mM to about 1000 mM, and the like.

The neutral hydroxylamine compounds of the invention and their concentrations as well as other conditions for the assays including temperature, pH, and contacting time are set forth above.

In additional embodiments of the invention, DNA glycosylase and neutral hydroxylamine can be used to detect clustered or closely spaced bistranded base damage and mismatch (clustered modified bases and/or mismatch). In such embodiments, a first portion of DNA sample is treated with a neutral hydroxylamine in which the true double stranded breaks are detected. A second portion of the DNA sample is treated with a neutral hydroxylamine and then with a DNA glycosylase and AP-lyase (e.g., Endonuclease III). Alternatively, the second portion of the DNA sample is treated with a neutral hydroxylamine and then with a DNA glycosylase without AP-lyase (e.g., uracil DNA glycosylase) followed by monoamine and/or polyamine treatment. The double stranded breaks are then detected in the second sample. The difference between the number of double stranded breaks detected in the first portion and the second portion represent the clustered modified bases or the clustered mismatch.

Accordingly, in some embodiments, methods are provided for detecting clustered or closely spaced bi-stranded base damage or mismatches in a DNA sample, the methods comprising: contacting a first portion of the DNA sample with a neutral hydroxylamine; detecting true double strand breaks in the DNA of the first portion; contacting the DNA of a second portion of the DNA sample with a neutral hydroxylamine; contacting the second portion of the DNA treated with the neutral hydroxylamine with a DNA glycosylase with AP-lyase; detecting double strand breaks in the DNA of the second portion; and comparing the number of double strand breaks detected in the DNA of the first portion of the DNA sample with the number of double strand breaks detected in the second portion of the DNA sample, wherein the number of closely space bi-strand base damage or mismatches in the DNA sample is represented by the number of double strand breaks detected in the second portion of the DNA sample that is greater than the number of double strand breaks detected in the first portion of the DNA; thus distinguishing between double strand breaks resulting from closely spaced bi-strand base damage or mismatches and double strand breaks resulting from any other cause in the DNA sample. In some embodiments, the neutral hydroxylamine is O-(tetrahydro-2H-pyran-2-yl)hydroxylamine (OTX).

Thus, DNA glycosylases useful with the present invention include those that generate AP sites and then cleave them by β-elimination. These DNA glycosylases include, but are not limited to, formamidopyrimidine-DNA glycosylase (FPG) and its homologues, E. coli endonuclease III (ENDIII) and its homologues, endonuclease VIII and its homologyues, endonuclease VIII-like protein and its homologues, 8-oxoguanine-DNA glycosylase (OGG1) and its homologues, and any combination thereof. The neutral hydroxylamine compounds of the invention and their concentrations as well as other conditions for the assays including temperature, pH, and contacting time and the like are set forth above.

In other embodiments of the invention, neutral hydroxylamines can be used in combination with DNA glycosylases and monoamines/polyamines to detect clustered modified bases (closely spaced bi-stranded base damage) and/or mismatches. Accordingly, methods are provided for detecting clustered or closely spaced bi-stranded modified bases or mismatches in a DNA sample, the methods comprising: contacting a first portion of the DNA sample with a neutral hydroxylamine; detecting true double strand breaks in the DNA of the first portion; contacting the DNA of a second portion of the DNA sample with a neutral hydroxylamine; then contacting the second portion of the DNA with a DNA glycosylase; then contacting the second portion of the DNA with a monamine and or polyamine; detecting double strand breaks in the DNA of the second portion; and comparing the number of double strand breaks detected in the DNA of the first portion of the DNA sample with the number of double strand breaks detected in the second portion of the DNA sample, wherein the number of closely space bi-strand modified bases or mismatches in the DNA sample is represented by the number of double strand breaks detected in the second portion of the DNA sample that is greater than the number of double strand breaks detected in the first portion of the DNA; thus distinguishing between double strand breaks resulting from closely spaced bi-strand modified bases or mismatches and double strand breaks resulting from any other cause in the DNA sample. In some embodiments, the neutral hydroxylamine is O-(tetrahydro-2H-pyran-2-yl)hydroxylamine (OTX).

Thus, DNA glycosylases useful with the present invention include those that generate AP sites but do not cleave AP sites. These DNA glycosylases include, but are not limited to, alkylbase DNA glycosylase, adenine-specific mismatch DNA glycosylase, 8-oxoguanine-DNA glycosylase (OGG1), uracil DNA glycosylase (UDG) and its homologues, and any combination thereof. The neutral hydroxylamine and monoamine/polyamine compounds of the invention and their concentrations as well as other conditions for the assays including temperature, pH, and contacting time are set forth above.

The DNA glycosylases of the present invention are provided at a concentration in a range from about 0.1 ng/μg DNA to about 10 μg/μg DNA. Thus, in some embodiments, the DNA glycosylase is provided at a concentration in a range from about 0.1 ng/μg DNA to about 1 μg/μg DNA, about 0.1 ng/μg DNA to about 100 ng/μg DNA, about 1 ng/μg DNA to about 10 μg/μg DNA, about 1 ng/μg DNA to about 1 μg/μg DNA, about 1 ng/μg DNA to about 100 ng/μg DNA, about 10 ng/μg DNA to about 10 μg/μg DNA, about 10 ng/μg DNA to about 1 μg/μg DNA, about 10 ng/μg DNA to about 100 ng/μg DNA and the like. In still other embodiments, the DNA glycosylase is provided at a concentration in a range from about 20 ng/μg DNA to about 40 ng/μg DNA. In further embodiments, the DNA glycosylase is provided at a concentration in a range from about 1 unit per μg DNA to about 100 units per μg DNA. Thus, in some embodiments, the DNA glycosylase is provided at a concentration in a range from about 5 units/μg DNA to about 20 units/μg DNA, about 5 units/μg DNA to about 40 units/μg DNA about 5 units/μg DNA to about 80 units/μg DNA, about 10 units/μg DNA to about 20 units/μg DNA, about 10 units/μg DNA to about 40 units/μg DNA, about 10 units/μg DNA to about 80 units/μg DNA, and the like. One unit is defined as the amount of enzyme that catalyzes the release of 60 pmol of uracil per minute from double-stranded, uracil-containing DNA in 30 minutes at 37° C.

The conditions for treatment of DNA with DNA glycosylase enzymes are well known in the art (Zielinska-Park et al. Carcinogenesis 25(9):1727-33 (2004)). In some embodiments of the invention, the treatment conditions include temperatures from about 4° C. to about 37° C. for about 1 min to about 24 hr. In particular embodiments, the conditions for treatment are a temperature from about 20° C. to about 37° C. for about 1 minute to about 2 hours.

As an example, the conditions for treatment of DNA with DNA glycosylases can be as follows: (1) for the DNA glycosylases FPG and MPG: 20-40 ng enzyme per μg DNA, 10 mM HEPES-KOH (pH 7.4), 100 mM KCl, 10 mM EDTA, and 0.1 mg/ml BSA) for 1 hour at 37°; (2) for the DNA glycosylase, UDG: 5-20 units per μg DNA, 20 mM Tris-HCl, 1 mM Dithiothreitol 1 mM EDTA pH 8.0 for 1 hour at 37° C.; (3) for EndoIII: 12.5 ng enzyme/ug DNA, 10 mM Tris-HCl buffer containing 1 mM EDTA and 100 mM NaCl for 30 min at 37° C. (PMID: 15117810); (4) for OGG1: 17.5 ng enzyme/ug DNA, 3 μg of bovine serum albumin, Tris-HCl/EDTA/NaCl. These conditions are set forth as exemplary conditions and are not intended to be limiting. Thus, other conditions known in the art can be used for the treatment of the DNA with the DNA glycosylase enzymes.

In other embodiments of the invention, a method is provided for more accurately detecting true DNA base damage or mismatches by DNA glycosylase treatment of a sample. The DNA single strand breaks resulting from treatment with DNA glycosylase can be used to indicate the presence of DNA base damage or mismatches (Duthie et al. Carcinogenesis 18:1709-1714 (1997)).

DNA glycolyases remove damaged bases or mismatches creating AP sites in the process. However, the true number of damaged DNA bases or mismatches is confounded by endogenous AP and oxidized AP sites. Thus, neutral hydroxylamines can be used to protect endogenous AP and oxidized AP sites prior to exposing the DNA or DNA in cells to the DNA glycosylase. The DNA treated with the neutral hydroxylamine can then be contacted with a DNA glycosylase, which creates new AP sites by removal of damaged bases or mismatches. These glycosylase-induced AP sites are measured as SSBs during an AGE or a comet assay. Because the endogenous AP and oxidized AP sites would be protected by the neutral hydroxylamine, base damage or mismatch can be estimated from the SSBs resulting from the cleaved glycosylase-induced AP sites. Thus, the combination of a neutral hydroxylamine-coupled SSB assay with the DNA glycosylase assay can provide a more accurate method for estimating DNA base damage (e.g., the amount of modified bases caused by alkylation, oxidation, deamination, etc) or mismatch.

Accordingly, further aspects of the present invention provide methods of detecting DNA base damage or mismatch in a DNA sample comprising: contacting the DNA of a first portion of the DNA sample with a neutral hydroxylamine; contacting the DNA with DNA glycosylase; denaturing the DNA; detecting single strand breaks in the denatured DNA of the first portion; contacting the DNA of a second portion of the DNA sample with a neutral hydroxylamine, denaturing the DNA and detecting the single strand breaks in the single strand breaks in the denatured DNA of the second portion, wherein the true number of damaged bases or mismatches in the DNA sample is represented by the number of single strand breaks detected in the first portion of the DNA sample that is greater than the number of single strand breaks detected in the second portion of the DNA; thus providing a true number of damaged bases or mismatches in the DNA sample. In some embodiments, the neutral hydroxylamine is O-(tetrahydro-2H-pyran-2-yl)hydroxylamine (OTX).

The neutral hydroxylamines and DNA glycosylases of the present invention and their concentrations as well as other conditions for the assays including temperature, pH, contacting time, and the like, are set forth above.

Other aspects of the invention provide methods of detecting DNA base damage or mismatch in DNA from a cell comprising: lysing the cell; denaturing the DNA from the lysed cell, wherein the DNA is contacted with a neutral hydroxylamine prior to and/or during the denaturing step; contacting the DNA with DNA glycosylase; and detecting single strand breaks in the DNA from the cell, whereby detecting single strand breaks detects DNA base damage or mismatch in the cell. In some embodiments, the neutral hydroxylamine is O-(tetrahydro-2H-pyran-2-yl)hydroxylamine.

In further embodiments of the invention, neutral hydroxylamine can be utilized to study DNA repair mechanisms in living cells. DNA repair occurs by the enzymatic removal of damaged bases or mismatches which then generates an AP site. The AP site results in a strand break or gap that can be filled by DNA polymerase and DNA ligase. Neutral hydroxylamine can react with the AP site without an adjacent nick or to the AP sites with an adjacent nick and inhibit DNA repair mechanisms in cells.

Neutral hydroxylamine provides advantages over other agents such as methoxyamine because of its larger size and its neutral pH. Thus, in contrast to methoxyamine, neutral hydroxylamine does not require high salt concentrations to neutralize the compound. Therefore, reducing or eliminating the effect of high salt concentrations on the cells. Acute elevation of NaCl inhibits the response to DNA damage, leading to inhibition of DNA repair and accumulation of DNA breaks through hypertonic stress (Demtrieva et al. Am. J. Physiol. Renal Physiol. 285: F266-F274 (2003); Demtrieva et al. Proc Natl Acad Sci USA 101(8):2317-2322 (2004); Demtrieva et al. Mutat. Res. 569(1-2):65-74 (2005)). Methoxyamine-HCl is frequently used for DNA repair inhibition to cultured cells at 8 mM to 30 mM. OTX can be utilized to inhibit DNA repair in cultured cells at much lower concentrations, such as, for example, 1 mM to 1.5 mM. Thus, under these conditions, methoxyamine solution after neutralization possesses 24 to 90 times higher levels of osmolarity compared to OTX solution. Cells are cultured tinder physiological osmotic conditions in order to test DNA repair function. Since OTX is neutral and does not need to generate salts during the chemical preparation, OTX provides a significant advantage over methoxyamine.

Further, while not wishing to be bound by any particular theory of the invention, it appears that the larger size of OTX may inhibit DNA repair by more efficiently disrupting protein function. Methoxyamine induces cell death at 20-30 mM, but OTX induces cell death at 2 mM in DT40 cells (chicken B-lymphocyte cell lines, unpublished data). Thus, neutral hydroxylamine provides an advantage for investigating the DNA repair pathway under physiological conditions as well as during stress conditions such as exposure to DNA damaging agents.

Accordingly, in some embodiments of the invention, methods are provided for investigating DNA repair mechanisms, the methods comprising contacting the one or more cell(s) with at least one agent which causes DNA damage or mismatch; contacting one or more cell(s) with a neutral hydroxylamine; and detecting single strand breaks; whereby detecting the single strand breaks detects failure of DNA to be repaired and failure of the DNA repair mechanisms.

In some embodiments of the invention, methods are provided for investigating DNA repair mechanisms, the methods comprising contacting the one or more cell(s) with at least one agent which causes DNA damage or mismatch; contacting one or more cell(s) with a neutral hydroxylamine; and detecting cell death; whereby detecting cell death detects failure of DNA to be repaired.

Agents which cause mismatch or DNA damage include, but are not limited to, alkylating agents, oxidizing agents, poly(ADP-Ribose) polymerase (PARP) inhibitor, poly(ADP-ribose) glycohydrolase inhibitor, topoisomerase I inhibitor, modified nucleotides, and any combination thereof.

In some embodiments of the invention, the neutral hydroxylamine is provided at a concentration in a range from about 0.1 mM to about 1000 mM, as described above. In some particular embodiments, the neutral hydroxylamine is provided at a concentration in a range from about 1.0 mM to about 2.0 mM. Accordingly, in some embodiments, the concentration of the neutral hydroxylamine is about 1.0 mM, 1.5 mM, 2.0 mM and the like. In particular embodiments of the present invention, the neutral hydroxylamine is O-(tetrahydro-2H-pyran-2-yl)hydroxylamine.

Alkylating agents of the present invention include, but are not limited to, methyl nitrosourea, ethyl nitrosourea and N-methyl-N′-nitro-N-nitrosoguanidine, N-ethyl-N′-nitro-N-nitrosoguanidine, MeOSO₂Et-lexitropsin, N-methylpyrrolecarboxamide, dimethyl sulfate, diethyl sulfate, methyl methanesulfonate, ethyl methanesulfonate, ethylbromide; diethyl sulfate; ethylmethane sulfonate; triethyloxonium tetrafluoroborate; alkyl sulfonate, including, but not limited to, busulfan, mannosulfan, treosulfan, and the like; nitrogen mustard including, but not limited to, cyclophosphamide, mechlorethamine, chlormethine, uramustine, melphalan, bendamustine, trofosfamide, chlorambucil, ifosfamide, and the like; hydrazine including, but not limited to, procarbazine; triazene including, but not limited to dacarbazine, temozolomide, and the like; aziridine including, but not limited to, carboquone, thiotepa, triaziquone, triethylenemelamine, and the like; ethyleneimine and methylmelamine including, but not limited to, hexamethylmelamine, thioptepa, and the like; nitrosurea including, but not limited to, carmustine, streptozocin and the like; mitomycin C; triazene including, but not limited to, dacarbazine; imidazotetrazine including, but not limited to, temozolomide; platinum based alkylating agents including, but not limited to, cisplatin, carboplatin, nedoplatin ocaliplatin, satraplatin, triplatin tetranitrate, and any combination thereof.

Oxidizing agents of the present invention include, but are not limited to, bleomycin, hydrogen peroxide, ultraviolet (UV) light, ionizing radiation, doxorubicin, and any combination thereof.

Nucleotide analogues of the present invention include, but are not limited to, 5-hydroxymethyl-2′-deoxyuridine, 5-iodo-2′-deoxyuridine, 5-fluorouracil, and any combination thereof.

PARP inhibitors of the present invention include, but are not limited to, 3-aminobenzamide, 3,4-dihydro-5[4-(1-piperindinyl)butoxy]-1(2H)-isoquinoline (DPQ), 5-Iodo-6-amino-1,2-benzopyrone, 1,5-Isoquinolinediol, 8-Hydroxy-2-methylquinazoline-4-one (NU 1025), 4-Amino-1,8-naphthalimide, 4H-Thieno[2,3-c]isoquinolin-5-one, and any combination thereof. Poly(ADP-ribose) glycohydrolase inhibitors of the present invention include, but are not limited to, adenosine 5′-diphosphate (hydroxymethyl)pyrrolidinediol, NH₄, and any combination thereof.

Topoisomerase I inhibitors of the present invention include, but are not limited to, camptothecin, topotecan, irinotecan, and any combination thereof.

The present invention is more particularly described in the Examples set forth below, which are not intended to be limiting of the embodiments of this invention.

### EXAMPLES

### Example 1

### Cell Lines and Cell Culture

The DT40 cells and isogenic DT40-derived Pol β null cells were grown in suspension in RPMI-1640 medium without phenol red (Invitrogen) and supplemented with 10% fetal bovine serum (heat inactivated; Sigma), 1% chicken serum, 100 μg/mL penicillin, and 100 μg/mL streptomycin (Ridpath et al., Cancer Res., 76, 11117-11122 (2007)). Cell lines were maintained at 39.5° C. and 5% CO₂.

### Example 2

### Chemical Exposure, Cell Harvest and DNA Isolation from Cultured Cells

DT40 and Pol β null cells were seeded into 100 cm dishes and allowed to divide overnight to reach the density of 1×10⁶ cells/mL. Cells were exposed to a continuous treatment of 1 mM methyl methanesulfonate (MMS; Aldrich) for up to 4 hours. Control cells received 1× phosphate buffered saline (PBS) for 0.5 hour. After incubation, cells were harvested, washed with chilled 1× PBS, pelleted, and stored at −80° C. until DNA isolation. Using this protocol, DT40 cells were also incubated in the absence of chemical to obtain a stock of untreated DNA.

DNA was extracted using a PureGene® DNA extraction kit (Gentra Systems. Inc.) with modifications as described previously (Nakamura et al., J. Biol. Chem., 275, 5323-5328 (2000)).

### Example 3

### AP Site Preparation by Heat/Acid Conditions

AP sites were produced in stock DT40 DNA through exposure to heat/acid conditions. DT40 DNA was incubated in sodium citrate buffer (10 mM sodium citrate, 10 mM NaH₂PO₄, 10 mM NaCl pH 5) for 5 minutes at 70° C., followed by rapid chilling on ice to stop the reaction (Nakamura et al., Cancer Res., 58, 222-225 (1998)). Depurinated DNA was collected using Microcon® YM-3 centrifugal filter devices (Amicon Bioseparations). This DNA was then washed several times with HEPES buffer (50 mM, pH 7) supplemented with 1 mM 2,2,6,6-tetramethylpiperidine-N-oxyl (TEMPO). DNA was resuspended in HEPES buffer containing 1 mM TEMPO.

### Example 6

### Creation of a pH Gradient through Treatment with NaOH

Heat/acid-treated DT40 DNA was exposed to various concentrations of NaOH (0.1-250 mM) in HEPES buffer (50 mM) at 37° C. for 15 minutes. The samples were isolated, washed, and resuspended as described above.

### Example 7

### Measurement of SSB Repair through NAD(P)H Depletion

SSB repair capacity was determined by a real-time colorimetric assay that measures intracellular NAD(P)H levels (Tano et al., DNA Repair, 6, 869-875 (2007)). PARP-1 activation during SSB repair leads to NAD⁺ consumption, which can be indirectly monitored through NAD(P)H depletion (Nakamura et al., Nucleic Acids Res., 31, e104 (2003)). Briefly, 5×10³ cells/well were seeded into a 96-well plate. After a 30 minute incubation period, the cells were treated with 1 mM MMS quickly followed by the addition of a dye containing XTT (Sigma) and 1-methoxy PMS (Sigma). For up to 4 hours, the NAD(P)H-dependent conversion of the XTT tetrazolium salt to a yellow colored formazan dye was measured spectrophotometrically. Decreases in intracellular NAD(P)H levels were determined through comparison of values to those of the PBS control.

### Example 8

### Measurement of AP Sites through an ARP-Slot Blot Assay

AP sites were measured using an aldehyde reactive probe (ARP; Dojindo Molecular Technology, Gaithersburg, Md., USA) coupled slot blot technique, previously described (Nakamura et al. (1998) Highly Sensitive Apurinic/Apyrimidinic Site Assay Can Detect Spontaneous and Chemically Induced Depurination under Physiological Conditions. Cancer Res., 58, 222-225.).

### Example 9

### Alkaline Gel Electrophoresis

An alkaline gel electrophoresis protocol (AGE) (Drouin et al., in: Technologies for Detection of DNA Damage and Mutations (G. P. Pfeifer, ed.). Plenum Press, New York, N.Y., pp. 37-43 (1996)) with modifications was used to assess SSBs and ALSs in DNA from exposed cells. The 0.7% agarose gel was prepared in gel buffer (50 mM NaCl, 1 mM EDTA, pH 7) and deionized water. After forming, the gel was soaked in mild alkaline running buffer (30 mM NaOH, 1 mM EDTA, pH 12.4) for at least 30 minutes. Equal amounts of DNA (3-5 μg) were incubated for 1 hour at 37° C. in the presence or absence of 30 mM OTX (Aldrich) in 50 mM HEPES buffer. Similar to the other neutral hydroxylamines, OTX does not require buffering before or during incubation with DNA. Following OTX exposure, the samples were denatured through treatment with an alkaline buffer (100 mM NaOH, 30 mM OTX, 50 mM HEPES, pH 12.8) for 20 minutes at 37° C. Samples then received loading buffer (10 mM NaOH, 95% formamide (Fluka), 0.05% bromophenol blue, 0.05% xylene cyanol) and were loaded onto the agarose gel. The gel was subjected to electrophoresis at a constant voltage (30 V) for 16 hours at 4° C. At the completion of electrophoresis, the gel was placed in neutralization buffer (400 mM Tris, pH 7.5) for 20 minutes at room temperature. Gels were stained with acridine orange (5 μg/mL) for 1 hour followed by continuous destaining in deionized water. DNA migration was visualized using an Image Station 440CF system (Kodak).

### Example 10

### SSB Formation at N7-Methylguanine Adducts through Heat/Piperidine Displacement

To visualize the conversion of MMS induced N7-methylguanine (N7-meG) adducts into SSBs, the AGE assay described above was modified with a piperidine treatment (Mattes et. al., Biochem. Biophys. Acta 868:71-76 (1986)). Equal amounts of DNA (25 μg) were incubated for 1 hour at 37° C. in the presence of 30 mM OTX (Aldrich) and 1 mM MMS or PBS in 50 mM HEPES buffer (pH 7). Treated DNA was washed and collected using microcon YM-3 centrifugal filter devices (Amicon Bioseparations). The treated DNA was then incubated with alkaline buffer (pH≧12.8) that contained either 100 mM NaOH or 1 M piperidine for 20 minutes at 37° C. This DNA was then washed and collected by centrifugal filter devices, and the concentrations determined by UV. DNA (3-5 μg) from each sample received loading dye and was loaded onto a 0.7% agarose gel prepared as previously described. Electrophoresis, neutralization, staining, and image analysis were performed as described above.

### Example 11

### Affect of pH on Cleavage of AP Sites

During DNA denaturation at high pH, intact AP sites, one of the major ALSs existing in genomic DNA, are nicked 3′ to the lesion by β-elimination (Friedberg et al., In: DNA Repair and Mutagenesis (2^{nd} edition), ASM Press, Washington, D.C., pp. 9-69 (2006)). To account for this occurrence, the ability of pH to cleave AP sites generated by heat/acid treatment of isolated, genomic DNA was addressed.

AP site containing DNA was exposed to heat/acid conditions for 5 min. to induce intact AP sites. The DNA was then exposed to various concentrations of NaOH to create a pH gradient (ranging in pH from 10 to greater than 13) at 37° C. prior to being loaded into an agarose gel and subject to electrophoreses under alkaline conditions (pH 12.4). The gels were stained with acridine orange and the image was obtained with a Kodak Image Station 440CF system.

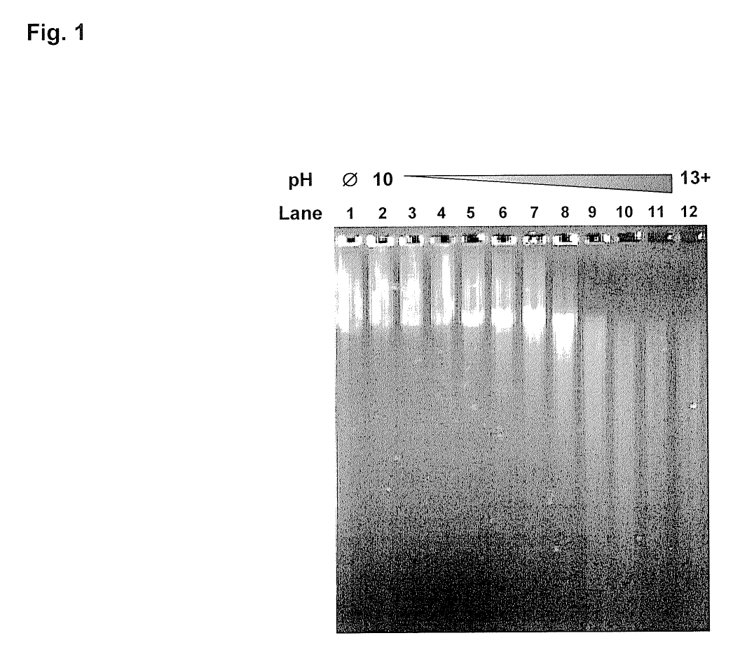

Concentrations of NaOH with pH levels below 11.9 demonstrated no major increase in DNA migration compared to the control sample, which received no NaOH exposure prior to electrophoresis (FIG. 1; Lanes 2-4 verses Lane 1). At a pH above 12.2, DNA migrated further with increasing NaOH concentration and pH level (FIG. 1; Lanes 5-12). It has been previously reported that AP sites are stable against alkaline cleavage at pH<12.6 (Kohn et. al. Pharmac. Ther. 49:55-77 (1991), Tice et. al., Environ. Mol. Mutagen. 35:206-221 (2000)). However, under the conditions used in this experiment, these findings indicate that AP sites generated by heat/acid conditions are stable at pH≦11.9 but are increasingly cleaved at pH≧12.2. In addition, large amounts of DNA were also observed immediately below the wells for lanes 1-8 (pH≦12.6). Those DNA samples could be incompletely denatured, thereby decreasing the rate of DNA migration during electrophoresis. Those DNA samples could be incompletely denatured, thereby decreasing the rate of DNA migration during electrophoresis. Though DNA denaturation and unwinding relies on the disruption of hydrogen bonds at pH>12 (Kohn et. al. Pharmac. Ther. 49:55-77 (1991), Tice et. al., Environ. Mol. Mutagen. 35:206-221 (2000)), the complete disruption of these bonds, and thus complete DNA denaturation, may be more efficient under stronger alkaline conditions. Based on the results of this experiment and concern regarding underestimation of SSB formation due to incomplete DNA denaturation under mild basic conditions (pH≦12.6), 100 mM NaOH (pH 12.8) was selected for DNA unwinding in subsequent experiments.

### Example 12

### O-(tetrahydro-2H-pyran-2-yl)hydroxylamine Optimization for Protection of AP Sites

With knowledge of optimal AP site cleavage (pH≧12.2) and DNA denaturation (pH≦12.56) conditions, the utility of stabilizing AP sites prior to complete unwinding at high pH was assessed for subsequent gel electrophoresis. O-hydroxyalamines, such as acidic methoxyamine and neutral ARP, efficiently react with AP and oxidized AP sites to generate very stable complexes, which are resistant to β-elimination from Pol β or high pH (Horton et al., J. Biol. Chem., 275:2211-2218 (2000); Nakamura et al., Cancer Res., 58:222-225(1998); Nakamura et al., Cancer Res., 59:2522-2526 (1999)). OTX, a hydroxylamine compound, was selected due not only to its commercial availability at reasonable cost, but also its simple preparation, which requires no titration to neutral pH, thereby eliminating high salt contamination. The optimal concentration of OTX needed to protect AP sites from alkaline scission during DNA denaturation was examined in genomic DNA exposed to heat/acid conditions. DNA containing intact AP sites was exposed to a concentration gradient of OTX (0.1-1000 mM) at 37° C. for 1 hour prior to denaturation with 100 mM NaOH (pH 12.8) and electrophoresis under mild alkaline conditions (pH 12.4). SSB formation from cleavage of the AP sites during alkali denaturation was observed as increased DNA migration during electrophoresis.

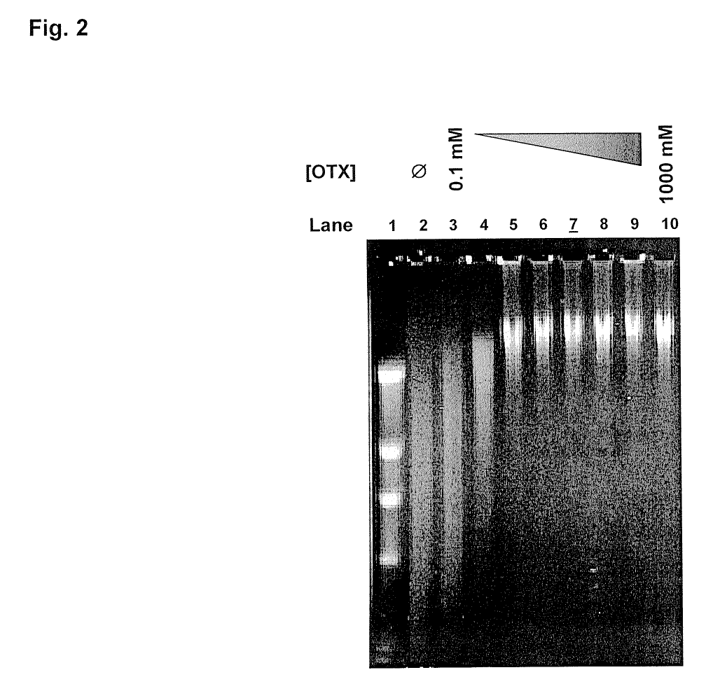

Protection from alkaline cleavage by the reaction of OTX with AP sites was observed as hindered DNA migration during electrophoresis. Little to no protection of AP sites from alkaline cleavage was observed when DNA was pretreated with concentrations of OTX<1 mM (FIG. 2; Lanes 3-4). Increased protection of AP sites from alkaline scission by OTX was observed between 1 to 3 mM (FIG. 2; Lanes 5-7) with complete protection achieved with concentrations of OTX≧10 mM (FIG. 2; Lanes 8-11). Based on these results, 30 mM OTX was deemed as the optimum concentration to protect AP sites from alkaline cleavage and was utilized for the remainder of the experiments.

### Example 13

### Accumulation of Single Strand Break Repair Intermediates in DT40 Cells Exposed to MMS Exposure

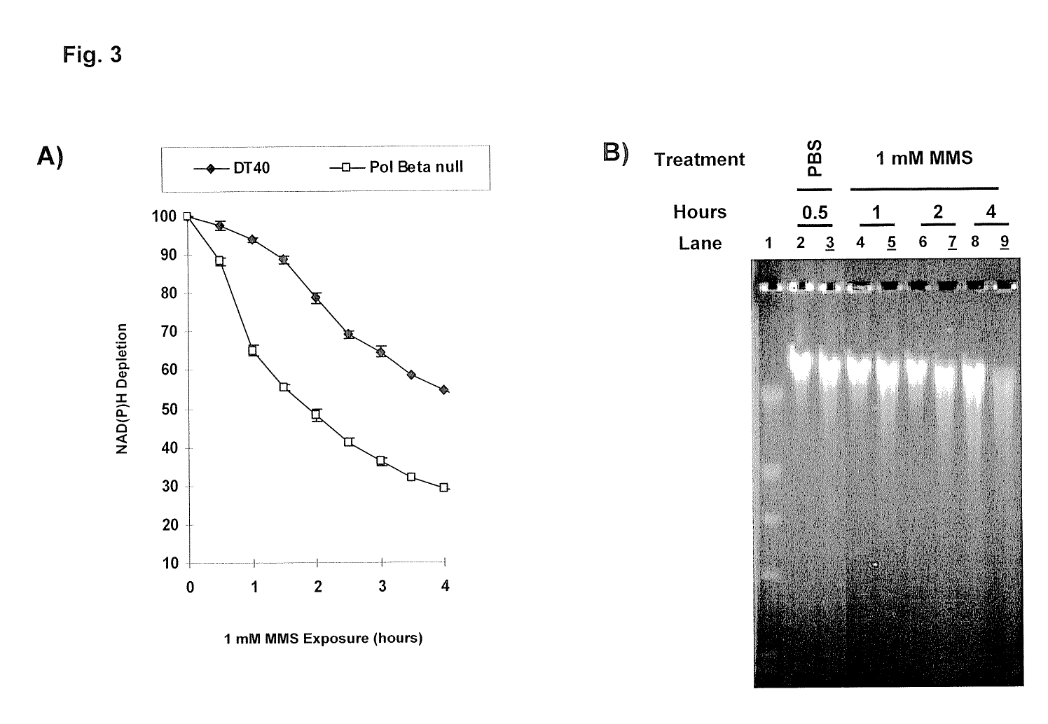

Established methods for detecting SSBs have not been adequately characterized for pH effects on DNA structure and chemistry and also lack a high specificity for SSBs, primarily because of artifactual SSB formation. Potentially, the majority of published data regarding SSB accumulation caused by genotoxic stress may be difficult to interpret due to unintended SSB formation caused by the incision of intact AP sites during strong alkaline conditions. Therefore, the OTX-coupled alkaline gel electrophoresis (OTX-AGE) was used to determine the accumulation of SSBs as intermediates during a BER response to an acute MMS exposure in the DT40 and Pol β null cells. A real-time assay based on NAD(P)H depletion was used to first established an accumulation of SSBs in the present cell system. Observations from the real-time assay were then compared with those obtained by OTX-AGE. The accumulation of SSBs leads to PARP-1 over-activation and the consumption of NAD+, which is indirectly monitored in living cells through the measurement of intracellular NAD(P)H depletion using tetrazolium salts, such as XTT, during chemical exposure (Tano et al., DNA Repair, 6, 869-875 (2007)). When exposed to 1 mM MMS for up to 4 hours, DT40 cells displayed a time-dependent decrease in intracellular NAD(P)H levels, suggesting an accumulation of SSBs (FIG. 3A). Similarly, the Pol β null cells displayed a time-dependent decrease but with more extensive depletion occurring as early as 30 minutes of MMS exposure (FIG. 3A). These NAD(P)H depletions were mostly protected by the PARP1 inhibitor 3-aminobenzamide. Therefore, NAD(P)H depletion in DT40 and Pol β null cells exposed to MMS was due to an accumulation of SSBs.

SSBs formed during the early stages of BER must be processed efficiently to limit the accumulation of these intermediates, which can cause them to potentially become more toxic and mutagenic (Kunz et al., Proc. Natl. Acad. Sci. USA, 16:8165-8169 (1994); Simonelli et al., Nucleic Acids Res., 33:4404-4411(2005)) than the preceding base damage; e.g., the non-toxic and non-mutagenic N7-methylguanine (Gates et al., Chem. Res. Toxicol., 17:839-856 (2004)) and the toxic N3-methyladenine (Varadarajan et al., Biochemistry, 42:14318-14327 (2003)).

During acute MMS exposure, the NAD(P)H data suggested that BER become uncoupled leading to accumulation of SSBs in both the wild-type and Pol β deficient cell lines. A greater accumulation of SSBs detected by more extensive NAD(P)H depletion in the Pol β null cells appears to be due to the inability of these cells to eliminate 5′-dRp by β-elimination (Sobol et al., Nature, 405, 807-810 (2000)) or later ligation (Ho et al., Nucleic Acids Res., 31, 7032-7040 (2003)). Based on the results from the NAD(P)H depletion assay described above, DNA was isolated from DT40 and Pol β null cells exposed to 1 mM MMS (1-4 hours) for the direct detection of SSBs by OTX-AGE. MMS (1 mM) caused a slight increase in SSBs in DT40 cells exposed from 1 to 2 hours and a marked induction of SSBs at 4 hours of exposure (FIG. 3B). In contrast, a clear time-dependent increase in DNA migration was observed in the Pol β null cell line indicating an increase in SSB accumulation during the 1 mM MMS exposure (FIG. 3B). The intensity of DNA fragmentation was slightly lower at 4 hours compared to the other time points in the Pol β null cells, probably due to extensive DNA fragmentation and migration in this sample from high SSBs levels. When comparing the cell lines, greater DNA migration over time was observed in the Pol β null cells indicating more SSB accumulation in this repair deficient cell line (FIG. 3B; Odd verses even lanes). These observations of the OTX-AGE are in agreement with those obtained by measuring NAD(P)H depletion during MMS exposure.

Interestingly, OTX-AGE visualization of SSBs in the cell lines during a control exposure to PBS demonstrated that the Pol β null cells had more endogenous SSBs than the repair proficient DT40 cells (FIG. 3B; Lanes 2-3). This difference in endogenous SSBs was not detectable through the NAD(P)H depletion assay. Separate OTX-AGE analysis of PBS exposed DT40 and Pol β null cells confirmed this difference in endogenous SSBs between the repair proficient and deficient cell lines (data not shown). Because BER corrects base damage caused by both exogenous and endogenous sources, a defect in this repair system can result in more SSB accumulation regardless of exposure.

### Example 14

### AP Site Formation in BER-Deficient DT40 Cells During MMS Exposure

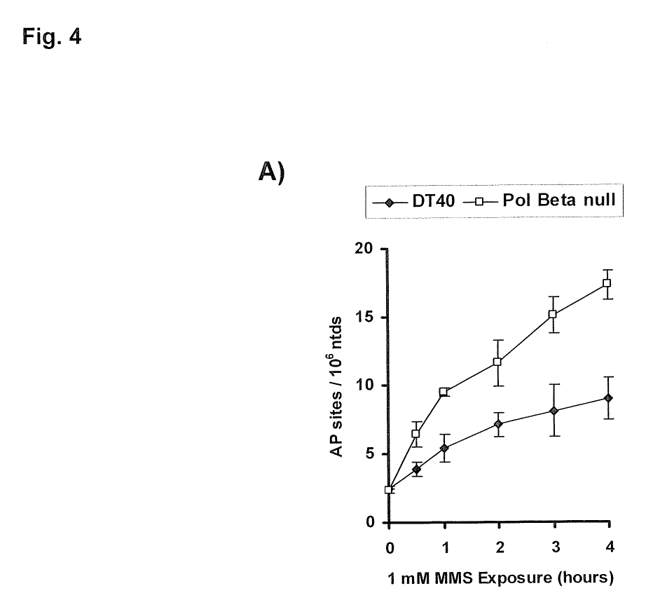

BER of alkylated bases leads to the formation of AP sites as pathway intermediates. The affect of Pol β status on the accumulation of AP sites during an acute MMS exposure was examined in DT40 and Pol β null cells using a quantitative slot-blot method (ASB assay) and the OTX-AGE assay. In the ASB assay, both cell lines displayed a time-dependent, significant (p<0.01 for all time points, student t-test) increase in AP sites during continuous exposure to 1 mM MMS compared to control. Furthermore, the Pol β null displayed a more massive AP site formation than the parental DT40 cells (FIG. 4A). The AP site data at 1 hour were different to what was detected in mouse embryonic fibroblasts deficient in Pol β (Sobol et al., Proc. Natl. Acad. Sci. USA. 99, 6860-6865 (2002)). This discrepancy can be explained by an improvement in DNA extraction through the use of the free radical scavenger TEMPO, which reduces artifactual induction of AP sites and also differences inherent between mammalian and avian cell lines.

The binding of aldehydic AP sites with a hydroxylamine, such as OTX, protects them from alkaline scission during AGE. Therefore, preparing DNA samples with and without OTX for AGE allows for the detection of SSBs only (i.e., with OTX treatment) or a combination of SSBs and AP sites (i.e., without OTX treatment). By comparing OTX treated and untreated pairs of DNA samples, a visualization of the extent of intact AP site formation can be obtained with AGE analysis. OTX treatment did not reveal a difference in DNA migration among the control samples of each cell line suggesting that there were low endogenous, intact AP site levels within each DT40 based cell line (FIG. 4B-C; Lanes 2-3). This observation is similar to the low levels of AP sites reported for control cells as determined by the ASB assay. After MMS exposure, AGE analysis of the DT40 DNA revealed a mild time-dependent increase in SSBs only (FIG. 4B; Lanes 4-11; even lanes), and also a stronger time-dependent increase in intact AP sites performed by AGE without OTX (FIG. 4B; Lane 4-11; odd lanes). A similar pattern was observed for MMS treated Pol β null cells, except that both SSB and intact AP site formation were greater in extent than wild-type cells (FIG. 4C; Lanes 4-11). These visual observations of greater intact AP site formation within the null cells were in agreement with the quantitative data obtained by the ASB assay.

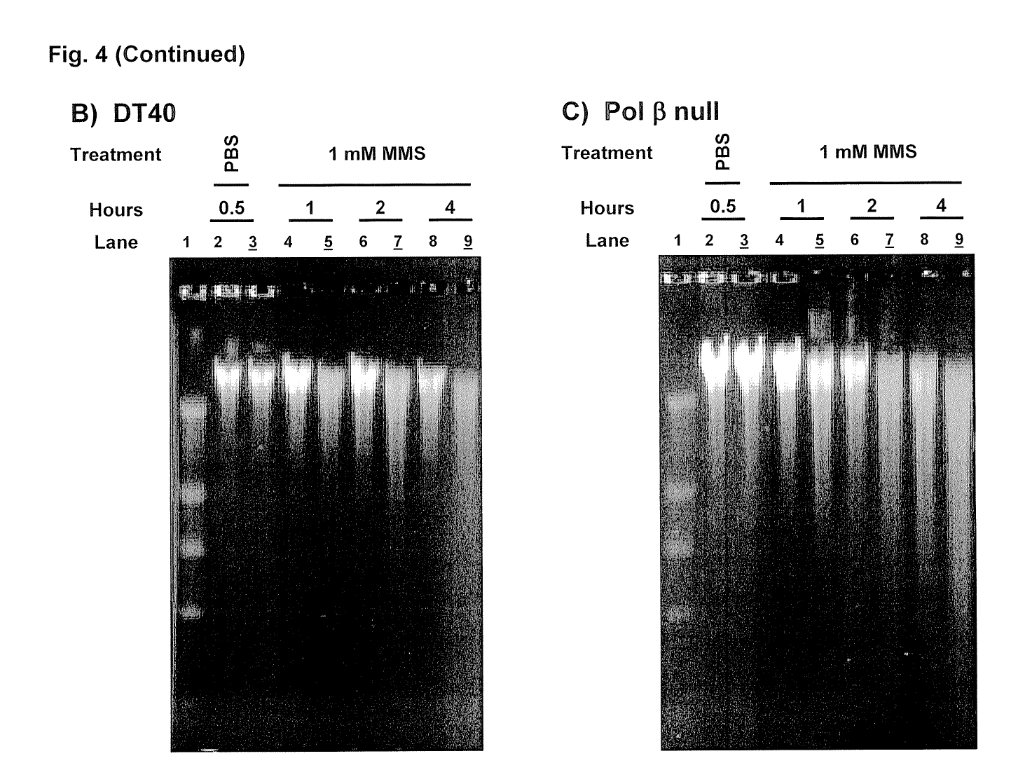

ALSs such as N7-methylguanine (N7-meG), the predominate lesion formed by monofunctional alkylating agents, are generated during MMS exposure (Wyatt et. al., Chem. Res. Toxicol. 19: 1580-1594 (2006)). In the absence of OTX, ALSs other than AP sites may contribute to SSBs detected during AGE. To assess this phenomenon, isolated, genomic DNA was simultaneously exposed to MMS (0 or 1 mM) and 30 mM OTX for one hour to induce N7-meG formation and protect pre-existing or endogenous aldehydic sites from alkaline strand scission during subsequent AGE. As a positive demonstration of the contribution of ALSs to the SSBs detected, treated DNA was then denatured with either 100 mM NaOH or 1 M piperidine for 20 minutes prior to being loaded into an agarose gel and subjected to electrophoresis under alkaline conditions (pH 12.4). The introduction of heat/piperidine and resulting alkaline pH induced SSBs at N7-meG adducts through a series of chemical events involving imidazole ring opening of N7-meG, displacement of the resulting formamido-pyrimidine from the deoxyribose backbone with AP site formation, and β-elimination of the AP site with concurrent SSB formation (Mattes et. al., Biochim. Biophys. Acta 868:71-76 (1986)). When the MMS exposed DNA received piperidine treatment, a notable increase in DNA migration was observed (FIG. 5; Lane 5); however, the increase in DNA migration was not seen in the PBS control that also received piperidine treatment (FIG. 5; Lane 4). For MMS treated and control DNA subjected to NaOH induced DNA denaturation, there was no difference in the amount of SSBs (FIG. 5; Lanes 2-3). Together, these data indicated that AP sites were the predominant source of ALSs in the highly methylated DNA analyzed under our conditions.

Overall, these results demonstrated that while the ASB assay provides an entire fraction of AP sites (i.e., intact AP sites and cleaved AP sites), the difference between AGE analysis with and without OTX defines the existence of intact AP sites before incision by either type I or type II AP endonuclease. These data also indicate that MMS causes the accumulation of not only overall AP sites, but also intact AP sites during the exposure of methylating agents, suggesting that incision of AP sites by AP endonuclease 1 can be saturated under massive DNA methylation by MMS.

### Example 15

### Advantages of the OTX-Coupled AGE Assay and Application to Other SSB Assays

In addition to being a specific method of detecting SSBs, the OTX-AGE assay offers a number of advantages. This approach is a relatively easy, time efficient assay based on common electrophoresis techniques. Because it is an electrophoretic method, the equipment used in the assay is often available in laboratories and the chemicals are readily available from commercial sources. Since the OTX-AGE assay uses very little DNA (3-5 μg per sample), this method can be used in situations where DNA sample amounts are limited. This assay is also versatile because samples analyzed by OTX-AGE may represent either DNA isolated from various tissues or cells after in vivo exposures or DNA purified after in vitro exposures to genotoxicants; this latter experimental design would not be applicable to the comet assay. The rapid generation of results from OTX-AGE is also very attractive. This method could serve as a valuable means for rapidly screening the ability of exogenous and endogenous agents to induce SSBs prior to assessing additional measures of DNA damage.

The use of OTX could also benefit other established alkaline-based SSB assays, such as the comet assay. Inclusion of OTX in the cell washing, lysis, and denaturation buffers for the comet assay would protect AP sites within cells during processing and electrophoresis. Lysis of the cells followed by complete DNA denaturation could then be achieved under high alkaline conditions without a reduction in assay sensitivity or specificity.

Thus, the present study reports the development and validation of a rapid and specific assay for the detection of SSBs. Based on agarose gel electrophoresis techniques, OTX provides protection of AP sites from alkaline strand scission while allowing for complete denaturation of DNA. Both aspects are needed for the specific and sensitive detection of real SSBs. The assay has been applied to DNA from MMS treated DT40 and Pol β null cells and the Pol β null cells displayed higher SSB formation. To our knowledge, this is the first time the accumulation of real SSBs, without potential confounding ALSs, has been demonstrated in BER deficient cells. The OTX-AGE assay offers many benefits and a similar approach may enhance other alkaline-based SSB detection methods through the use of OTX.

### Example 16

### OTX-Coupled Comet Assay

Chinese hamster ovary (CHO) AA8 cells were seeded at a concentration 0.5×10⁶ cell/mL into several 10 cm culture dishes with 10 mL of MEM-α media (Gibco) supplemented with 10% fetal bovine serum (heat inactivated; Sigma), 100 μg/mL penicillin, and 100 μg/mL streptomycin. These were allowed to grow for 48 hours at 37° C. and 5% CO₂. Culture dishes were about 80-85% confluent at harvest.

Before cells were harvested for use in the comet assay, the lysis solution (2.5 M NaCl, 100 mM Na2-EDTA, 10 mM Trizma, pH 10) was completed with the addition of 10% (v/v) DMSO and 1% (v/v) Triton X-100, mixed thoroughly, and refrigerated at 4° C. A 0.5% solution of low melting point agarose (LMPA) was also prepared and allowed to equilibrate to 37° C. in a water bath. Partially frosted microscope slides, pre-coated several days before with 50 μL of 1% normal melting point agarose (NMPA), were labeled and set aside.

Prior to cell harvest, the media was removed and each dish was washed with 10 mL of 1×PBS. An additional 2 mL of 1×PBS was added to each dish and the cells were harvested using a rubber policeman. Harvested cells were combined into 15 mL centrifuged tubes and gently mixed by pipetting to obtain a homogenous cell sample. The cell count was obtained using a hemacytometer. The volume of cell sample needed to obtain 11,000 cells per slide to be prepared was added to a new 15 mL centrifuge tube and centrifuged for 5 minutes at 1,100 RPM. The supernatant was removed and the cell pellet resuspended in 1×PBS at a volume equal to 10 μL per slide to be prepared (e.g. 5 slides×10 μL per slide=50 μL 1×PBS total).

For each slide to be prepared, 10 μL of the cell sample was added to a 1.5 mL centrifuge tube. The cell layer of the slide was created by first gently mixing 100 μL of the 0.5% LMPA with the 10 μL of cell sample in the tube. This mixture was then added to a pre-coated slide and a coverslip placed onto the slide to create a thin layer of the cell mixture. Slides with cell layers were then refrigerated at 4° C. for 15 minutes to solidify the gel. After this incubation, the initial coverslips were removed and an additional 150 μL of 0.5% LMPA was added to each slide. A fresh coverslip was placed onto each slide to create a thin top layer and the slides were refrigerated for another 15 minutes. After removing the coverslip, completed slides that were receiving MMS treatment were placed in a slide chamber containing warmed MEM-α media with 1 mM MMS for 10 minutes. The slide chamber was kept warm by placing it within an incubator held at 37° C. After the 10 minutes, the slides were washed with 1×PBS for 5 minutes at room temperature and then transferred to slide chambers containing the chilled lysis solution. Completed slides that were not receiving MMS treatment were placed immediately into chilled lysis solution once the top gel layer had solidified. All slides were incubated at 4° C. in lysis solution for 18 hours. After all the slides were place in lysis solution, the alkaline electrophoresis buffer (300 mM NaOH, 1 mM Na₂-EDTA, pH>13) was prepared and allowed to refrigerate at 4° C. overnight.

After 18 hours, the slides were placed in 400 mM Trizma (pH 7.5) for 5 minutes at room temperature to remove any residual lysis solution. Slides that were to receive OTX protection were then placed into slide chambers containing 300 mM OTX (pH 7.5), while those not receiving OTX protection were placed in chambers containing 400 mM Trizma (pH 7.5). All slide chambers were placed in an incubator held at 37° C. Incubation times in either OTX or Trizma varied from 0-4 hours.

At the end of incubation, the slides were again washed in 400 mM Trizma (pH 7.5) for 5 minutes at room temperature then placed in an electrophoresis chamber. Chilled alkaline electrophoresis buffer was added to the chamber and circulated. The slides were incubated in the circulating buffer for 20 minutes to allow the DNA to unwind. After this incubation period, the power source was turned on and the volume of buffer adjusted so that electrophoresis was run at a constant 300 mA and 25 V for 45 minutes. Slides were then removed from the alkaline buffer and neutralized in 400 mM Trizma (pH 7.5) for 10 minutes at room temperature. Slides were prepared for storage by dehydrating (drying) the gel in 100% ethanol. The slides were then stored at room temperature in a dry place.

For analysis, the slides were rehydrated with 60 μL of 1× ethidium bromide. Images of 50 cells per slide are captured using an inverted Olympus microscopy equipped with fluorescence and a camera. These images are then analyzed using CometScore software (TriTek Corporation, Sumerduck, Va.) and the tail moments compared among the sample groups.

### Results

The results are based on 50 cell images per sample group and statistics were calculated using InStat software (GraphPad Software, Inc, San Diego, Calif.) with P<0.05 was considered significant.

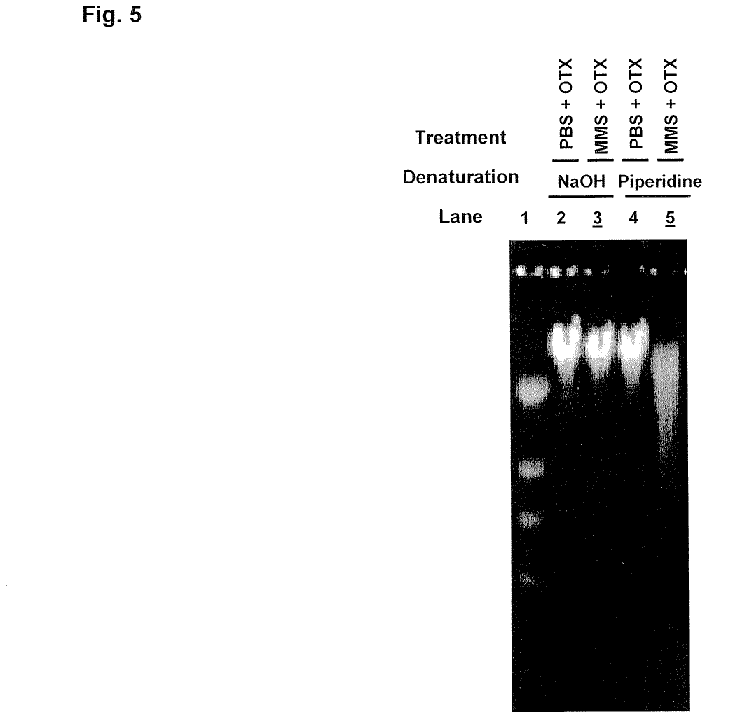

The difference in tail moments resulting from MMS exposure are shown in FIG. 6. A 10 minute exposure to 1 mM MMS resulted in a significant difference in tail moment when compared to the untreated control (no MMS).

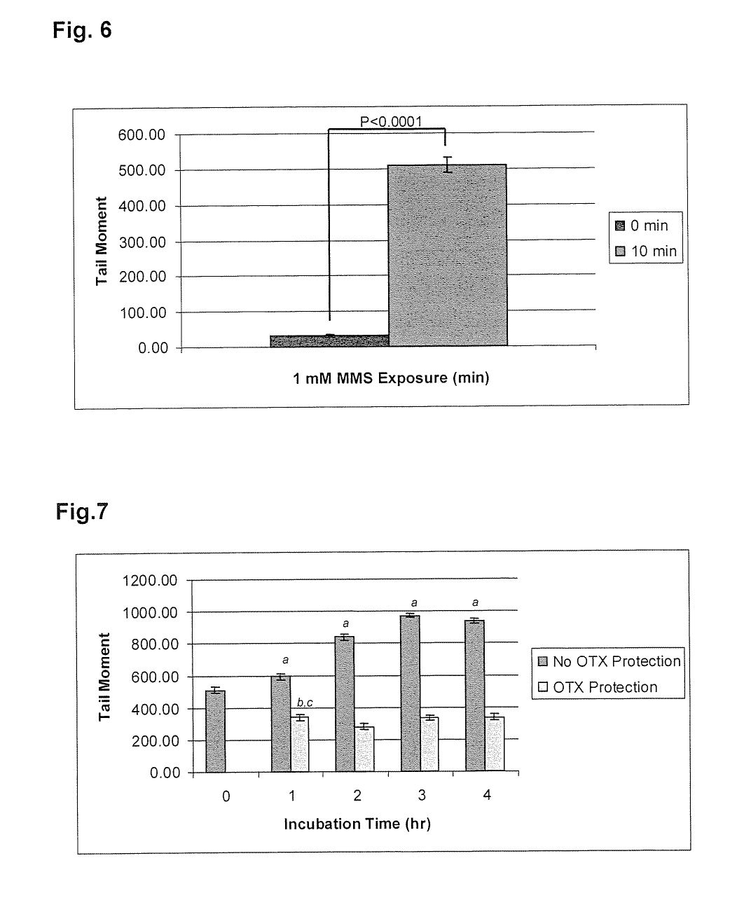

The difference in tail moments resulting from MMS exposure with and without OTX Protection is shown in FIG. 7. After a 10 minute exposure to 1 mM MMS followed by overnight lysis, slides were incubated in the absence or presence of 300 mM OTX for 0-4 hours prior to DNA unwinding and electrophoresis. Slides that did not receive OTX protection (i.e. tail moments represented by the green bars) displayed a significant increase in tail moment as the incubation time increased. This suggests that artifactual damage (i.e. single strand breaks, apurinic/apyrimidinic sites) was induced during the processing of the slides. In comparison, the slides that did receive OTX protection (i.e. tail moments represented by the yellow bars) displayed no significant increase in tail moments over the various incubation times. This lack of increase over time suggests that OTX protected against the induction of artifactual damage during the processing of the slides. It also suggests that a one hour incubation with OTX is sufficient for OTX protection.

Specifically looking at the 1 hour incubation samples, the data showed a significant decrease in tail moment with OTX protection. This suggests that OTX can provide protection against alkaline-induced AP site cleavage, which leads to artifactual SSB detection. A significant decrease in tail moment was also observed when comparing the one hour incubation sample that received OTX protection with the zero hour incubation control that did not receive OTX protection. Thus, as observed from the tail moments of samples that did not receive OTX, it appears that additional damage accumulates as the incubation time increases. This is damage that was not present in the cell at the start of the assay. Thus, this data indicates that artifactual SSBs can also arise during slide processing leading to higher tail moments that are not representative of the true level of damage in the cell prior to the start of the assay. The significant difference in the tail moments of the 1 hour samples that did or did not receive OTX suggests that OTX helps prevent artifactual SSBs from cleaved AP sites. The significant difference in the tail moments of the 1 hour sample that received OTX and the O hour control samples suggest that not only did OTX protect against artifactual SSBs arising form cleaved AP sites but also artifactual SSB arising from the processing of the slides.

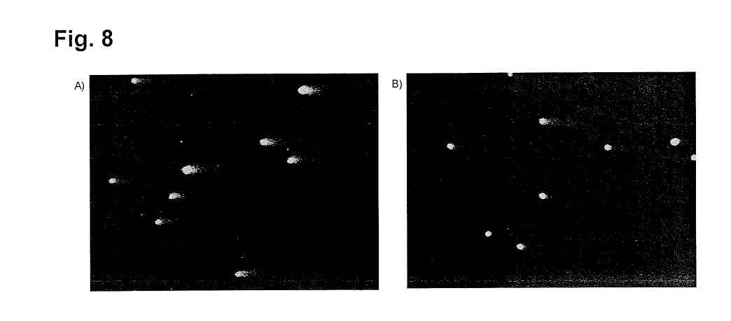

The visual difference in comets resulting from MMS exposure with and without OTX protection is shown in FIGS. 8A and 8B. A uniform comet appearance was observed in samples that were exposed to MMS without additional OTX protection (FIG. 8A). This uniform appearance was not observed in samples that were exposed to MMS with additional OTX protection (FIG. 8B). When looking at the population of cells on a slide as a whole, the addition of OTX provided significant protection leading to a decrease in tail moment (FIG. 7).

### Example 17

### Detection of in vivo DNA Damage in Cells

Detection of DNA strand breaks. Cells (˜1 million cells/ml medium) are treated with DNA damaging agents or DNA repair protein inhibitor plus or minus 0.1-5 mM OTX. OTX is added before (2-4 hours prior), during, and/or after exposure to DNA damaging agents. After treatment, cells are washed and fresh medium are added in the presence or absence of OTX. The cells are then harvested for detection of DNA single and double strand breaks.

Cell survival (colony formation assay). Cells (2000/dish) are plated, adhered for 18 h, and treated with DNA damaging agents or DNA repair protein inhibitor plus or minus 0.1-5 mM OTX. A OTX is added before (2-4 hours prior), during, and/or after exposure to DNA damaging agents. After treatment, cells are washed and fresh medium is added in the presence or absence of OTX. The cells are grown for a further 7-14 days prior to staining with methylene blue for determination of colonies containing more than 50 cells. Comparisons of drug-induced cytotoxicity consisted of a calculation of LC₅₀ of DNA damaging agents in the presence or absence of OTX. The degree of potentiation of DNA damaging agent by OTX is indicated by LC₅₀ for DNA damaging agent alone/LC₅₀ for DNA damaging agent plus OTX.

Cell survival (microplate-based cell survival assay). Cells (2000/well/250 uL medium, 24-well plate) are plated, and treated with DNA damaging agents or DNA repair protein inhibitor plus or minus 0.1-5 mM OTX. OTX is added before (2-4 hours prior), during, and/or after exposure to DNA damaging agents. After treatment, if necessary, cells are washed and fresh medium are added in the presence or absence of OTX. The cells are grown for a further 7 days prior to staining with tetrazolium salt, such as XTT, for determination of number of cells in each well. Comparisons of drug-induced cytotoxicity consists of a calculation of LC₅₀ of DNA damaging agents in the presence or absence of OTX. The degree of potentiation of DNA damaging agent by OTX is indicated by a LC₅₀ for DNA damaging agent alone/LC₅₀ for DNA damaging agent plus OTX.

The above examples clearly illustrate the advantage of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference into this application in order to more fully describe the state of the art to which this invention pertains.

That which is claimed is:

1. A method of detecting single strand breaks in a DNA sample comprising:
contacting the DNA with a neutral hydroxylamine;
denaturing the DNA; and
detecting single strand breaks in the denatured DNA.

2. The method of claim 1, wherein the neutral hydroxylamine is selected from the group consisting of 2-(2-(2-aminooxy-ethoxy)-ethoxy)-ethyl-hydroxylamine picrate. O-trityhydroxylamine, O-(tert-butyldimethylsilyl)hydroxylamine. O-(trimethylsilyl)hydroxylamine, O-(tetrahydro-2H-pyran-2-yl)hydroxylamine and aldehyde reactive probe.

3. The method of claim 1, wherein the hydroxylamine is O-(tetrahydro-2H-pyran-2-yl)hydroxylamine (OTX).

4. The method of claim 1, wherein the neutral hydroxylamine is provided at a concentration of about 3 mM to about 100 mM.

5. The method of claim 1, wherein the contacting comprises contacting the DNA with the neutral hydroxylamine at a temperature in a range from about 4° C. to about 40° C., a pH in a range from about 7.2 to about 7.6, and for a period of time in a range from about 30 min. to about 2 hours.

6. The method of claim 1, wherein the denaturing of the DNA occurs at a pH≧12.0.

7. A method of detecting single strand breaks in a DNA sample comprising:
contacting the DNA with O-(tetrahydro-2H-pyran-2-yl)hydroxylamine (OTX);
denaturing the DNA at a pH≧12.0; and
detecting single strand breaks in the denatured DNA.

8. The method of claim 7, wherein the OTX is provided at a concentration of 3 mM to 100 mM.

9. The method of claim 7, wherein the contacting comprises contacting the DNA with the hydroxylamine at a temperature in a range from about 4° C. to about 40° C., a pH in a range from about 7.2 to about 7.6, for a time period of about 30 min. to 2 hours.

10. A method of detecting single strand breaks in DNA from a cell comprising:
lysing the cell;
denaturing the DNA from the lysed cell, wherein the DNA is contacted with a neutral hydroxylamine prior to and/or during the denaturing step; and
detecting single strand breaks in the DNA from the cell.

11. The method of claim 10, wherein the neutral hydroxylamine is selected from the group consisting of 2-(2-(2-aminooxy-ethoxy)-ethoxy)-ethyl-hydroxylamine picrate, O-trityhydroxylamine, O-(tert-butyldimethylsilyl)hydroxylamine, O-(trimethylsilyl)hydroxylamine, O-(tetrahydro-2H-pyran-2-yl)hydroxylamine and aldehyde reactive probe.

12. The method of claim 10, wherein the neutral hydroxylamine is O-(tetrahydro-2H-pyran-2-yl)hydroxylamine (OTX).

13. The method of claim 10, wherein the neutral hydroxylamine is provided at a concentration of 3 mM to 300 mM.

14. The method of claim 10, wherein the lysing of the cell and the denaturing of the DNA occurs in the presence of the neutral hydroxylamine.

15. The method of claim 7, wherein the denaturing of the DNA occurs at a pH≧12.0.

16. A method of detecting single strand breaks in DNA from a cell comprising:
lysing the cell;
denaturing the DNA from the lysed cell, wherein the DNA is contacted with O-(tetrahydro-2H-pyran-2-yl)hydroxylamine (OTX) prior to and/or during the denaturing step and the denaturing of the DNA occurs at a pH≧12.0; and
detecting single strand breaks in the DNA from the cell.

17. The method of claim 16, wherein the OTX is provided at a concentration of 3 mM to 300 mM.

18. The method of claim 16, wherein the hydroxylamine is included in the lysing of the cell and the denaturing of the DNA.

19. A method of distinguishing between true single strand breaks and apurinic/apyrimidinic (AP) and oxidized AP sites in a DNA sample comprising:
contacting a first portion of the DNA sample with a neutral hydroxylamine;
denaturing the DNA of the first portion;
detecting single strand breaks in the denatured DNA of the first portion;
denaturing the DNA of a second portion of the DNA sample, wherein the second portion is not contacted with a neutral hydroxylamine;
detecting single strand breaks in the denatured DNA of the second portion; and
comparing the number of single strand breaks detected in the DNA of the first portion of the DNA sample with the number of single strand breaks detected in the second portion of the DNA sample,
wherein the number of true single strand breaks in the DNA sample is represented by the number of single strand breaks detected in the first portion of the DNA sample; and
the number of AP and oxidized AP sites present in the DNA sample is represented by the number of single strand breaks detected in the second portion of the DNA sample that is greater than the number of single strand breaks detected in the first portion of the DNA sample, thus distinguishing between true single strand breaks and AP and oxidized AP sites in the DNA sample.

20. The method of claim 19, wherein the neutral hydroxylamine is selected from the group consisting of 2-(2-(2-aminooxy-ethoxy)-ethoxy)-ethyl-hydroxylamine picrate, O-trityhydroxylamine, O-(tert-butyldimethylsilyl)hydroxylamine, O-(trimethylsilyl)hydroxylamine, O-(tetrahydro-2H-pyran-2-yl)hydroxylamine and aldehyde reactive probe.

21. The method of claim 19, wherein the neutral hydroxylamine is O-(tetrahydro-2H-pyran-2-yl)hydroxylamine (OTX).

22. The method of claim 19, wherein the neutral hydroxylamine is provided at a concentration of 3 mM to 100 mM.

23. The method of claim 19, wherein the contacting comprises contacting the DNA with the neutral hydroxylamine at a temperature in a range from about 4° C. to about 40° C., a pH in a range from about 7.2 to about 7.6, and for a period of time in a range from about 30 min. to about 2 hours.

24. The method of claim 19, wherein the denaturing of the DNA occurs at a pH≧12.0.

25. A method of distinguishing between true single strand breaks and apurinic/apyrimidinic (AP) sites in a DNA sample comprising:
contacting a first portion of the DNA sample with O-(tetrahydro-2H-pyran-2-yl)hydroxylamine (OTX);
denaturing the DNA at a pH≧12.0;
detecting single strand breaks in the denatured DNA of the first portion;
denaturing the DNA of a second portion of the DNA sample at a pH≧12.0, wherein the second portion is not contacted with OTX;
detecting single strand breaks in the denatured DNA of the second portion; and
comparing the number of single strand breaks detected in the DNA of the first portion of the DNA sample with the number of single strand breaks detected in the second portion of the DNA sample,
wherein the number of true single strand breaks in the DNA sample is represented by the number of single strand breaks detected in the first portion of the DNA sample; and
the number of AP sites present in the DNA sample is represented by the number of single strand breaks detected in the second portion of the DNA sample that is greater than the number of single strand breaks detected in the first portion of the DNA sample, thus distinguishing between true single strand breaks and apurinic/apyrimidinic (AP) sites in the DNA sample.

26. The method of claim 25, wherein the OTX contacting the first portion of the DNA sample is provided at a concentration of 3 mM to 100 mM.

27. The method of claim 25, wherein the contacting comprises contacting the DNA with the OTX at a temperature in a range from about 4° C. to about 40° C., a pH in a range from about 7.2 to about 7.6, for a time period of about 30 min. to 2 hours.

28. A method of distinguishing between true single strand breaks and AP and oxidized AP sites in DNA from a cell sample comprising:
lysing a first cell of the cell sample;
denaturing the DNA from the first cell, wherein the DNA is contacted with a neutral hydroxylamine prior to and/or during the denaturing step;
detecting single strand breaks in the DNA of the first cell of the cell sample,
lysing a second cell of the cell sample;
denaturing the DNA from the second cell wherein the DNA is not contacted with a neutral hydroxylamine;
detecting single strand breaks in the DNA of the second cell of the cell sample, and
comparing the number of single strand breaks detected in the DNA of the first cell of the cell sample with the number of single strand breaks detected in the DNA of the second cell of the cell sample in which the cell was not contacted with a neutral hydroxylamine,
wherein the number of true single strand breaks in the DNA of the cell(s) of the cell sample is represented by the number of single strand breaks detected in DNA of the first cell of the cell sample; and
the number of AP and oxidized AP sites in the DNA of the cell(s) of the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized AP sites in the DNA sample.

29. The method of claim 28, wherein the neutral hydroxylamine is selected from the group consisting of 2-(2-(2-aminooxy-ethoxy)-ethoxy)-ethyl-hydroxylamine picrate, O-trityhydroxylamine, O-(tert-butyldimethylsilyl)hydroxylamine, O-(trimethylsilyl)hydroxylamine, O-(tetrahydro-2H-pyran-2-yl)hydroxylamine and aldehyde reactive probe.

30. The method of claim 28, wherein the neutral hydroxylamine is O-(tetrahydro-2H-pyran-2-yl)hydroxylamine (OTX).

31. The method of claim 28, wherein the neutral hydroxylamine is provided at a concentration of 3 mM to 300 mM.

32. The method of claim 28, wherein the lysing of the cell and the denaturing of the DNA of the first portion of the cell sample occurs in the presence of the neutral hydroxylamine.

33. The method of claim 28, wherein the denaturing of the DNA occurs at a pH≧12.0.

34. A method of distinguishing between true single strand breaks and AP and oxidized AP sites in DNA from a cell sample comprising:
lysing a first cell of the cell sample;
denaturing the DNA from the first cell at a pH≧12.0, wherein the DNA is contacted with O-(tetrahydro-2H-pyran-2-yl)hydroxylamine (OTX) prior to and/or during the denaturing step;
detecting single strand breaks in the DNA of the first cell of the cell sample,
lysing a second cell of the cell sample;
denaturing the DNA from the second cell at a pH≧12.0, wherein the DNA is not contacted with a neutral hydroxylamine;
detecting single strand breaks in the DNA of the second cell of the cell sample, and
comparing the number of single strand breaks detected in the DNA of the first cell of the cell sample with the number of single strand breaks detected in the DNA of the second cell of a the cell sample in which the cell was not contacted with OTX,
wherein the number of true single strand breaks in the DNA of the cell(s) from the cell sample are represented by the number of single strand breaks detected in DNA of the first cell of the cell sample; and
the number of AP and oxidized AP sites in the DNA of the cell(s) from the cell sample are represented by the number of single strand breaks detected in the DNA of the second cell of the cell sample that is greater than the number of single strand breaks detected in the DNA of the first cell of the cell sample, thus distinguishing between true single strand breaks and AP and oxidized AP sites in DNA from the cell sample.

35. The method of claim 34, wherein the OTX is provided at a concentration of 3 mM to 300 mM.

36. The method of claim 34, wherein the lysing of the first cell and the denaturing of the DNA of the first cell occurs in the presence of OTX.
